(19) United States
(12) Patent Application Publication
(10) Pub. No.: US 2023/0257788 A1
(43) Pub. Date: Aug. 17, 2023

(54) OLIGONUCLEOTIDE ASSEMBLY USING HAIRPINS AND INVADING STRANDS

(71) Applicant: MICROSOFT TECHNOLOGY LICENSING, LLC, Redmond, WA (US)

(72) Inventors: Yuan-Jyue CHEN, Seattle, WA (US); Bichlien Hoang NGUYEN, Seattle, WA (US); Karin STRAUSS, Seattle, WA (US)

(21) Appl. No.: 17/670,350

(22) Filed: Feb. 11, 2022

(51) Int. Cl. C12P 19/34; C12N 15/11; B01L 3/00

(52) U.S. Cl. CPC C12P 19/34; B01L 3/502715; B01L 3/502761; C12N 15/11; B01L 2300/0645; C12N 2310/531

(57) ABSTRACT

Sequential assembly of oligonucleotide hairpins is used to create oligonucleotides that encode a specific sequence of arbitrary information. Each oligonucleotide hairpin includes a payload region in the loop region of the hairpin. The payload region encodes arbitrary information such as a binary digit. Overhang regions on the oligonucleotide hairpins hybridize to anchor strands attached to a substrate. The hybridized oligonucleotide hairpins are covalently attached to the anchor strands by ligase. Invading strands are used to open the hairpin structures by also hybridizing to the anchor strand and displacing the double-stranded stem region of the hairpin. This process is repeated with another oligonucleotide hairpin that hybridizes to the end of the previously added oligonucleotide hairpin. A microelectrode array may be used to control the location of hybridization and create multiple different oligonucleotides in parallel. Fully assembled oligonucleotides may be separated from the substrate and stored or otherwise processed.

### BACKGROUND

De novo synthesis of oligonucleotides has many applications including data storage. Synthetic oligonucleotides, such as deoxyribonucleic acid (DNA), can be used to store digital information with a much higher density and greater longevity than conventional media. Examples of data storage and other information technology applications for synthetic DNA are discussed in Meiser, L.C., Nguyen, B.H., Chen, YJ. et al. Synthetic DNA applications in information technology. Nat Commun 13, 352 (2022).

The vast majority of artificially synthesized oligonucleotides are created by chemical synthesis using the phosphoramidite process. This process involves multiple steps and is performed using the organic solvent acetonitrile. However, the phosphoramidite process is complex and creates waste that can be hazardous and expensive to process.

Oligonucleotides may also be synthesized with a template-independent DNA polymerase called terminal deoxynucleotidyl transferase (TdT). Enzymatic synthesis addresses some of the deficiencies of the phosphoramidite process. However, this enzyme can repeatedly add the same nucleotide multiple times creating unintended homopolymers. A variety of techniques have been identified to limit homopolymer creation, but each increases complexity and comes with its own set of drawbacks.

Alternative ways of creating oligonucleotides with specific, controllable sequences will be useful for information technology and other applications. The following disclosure is made with respect to these and other considerations.

### SUMMARY

This disclosure provides methods, oligonucleotide structures, and devices for assembling oligonucleotides by repeated hybridization of oligonucleotide hairpins to anchor strands. A substrate coated with single-stranded oligonucleotide anchor strands is contacted with an oligonucleotide hairpin. Each oligonucleotide hairpin has a stem region, a loop region, and an overhang region that extends from the stem region. Sequences of the oligonucleotide hairpins and the anchor strands are designed so that the overhang regions hybridize to the anchor strands. Ligase is used to form a nucleotide backbone between the hybridized oligonucleotide hairpins and ends of the anchor strands. The oligonucleotide hairpins are then opened by the introduction of an invading strand that uses strand displacement to separate the double-stranded stem region and open the oligonucleotide hairpins. This process is repeated with the addition of subsequent oligonucleotide hairpins that each hybridize and then are ligated to the ends of the previously added oligonucleotide hairpin.

The loop region of the oligonucleotide hairpins contains a payload region. The payload region may encode any arbitrary information such as a bit (“0” or “1”), a character (A, B, C, D, ...), or any other value. The order in which oligonucleotide hairpins are added creates an oligonucleotide that encodes a specific string of arbitrary information. After an oligonucleotide having the intended sequence of payload regions is created, it may be released from the substrate and stored or used for another information technology application.

Multiple oligonucleotides with different sequences of payload regions may be created in parallel by using a microelectrode array as the substrate. Selective activation of electrodes in the microelectrode array creates localized electric fields that electrostatically attract the oligonucleotide hairpins to specific locations on the surface of the substrate. Oligonucleotide hairpins hybridize to the anchor strands in proximity to the activated electrodes. The location of oligonucleotide hybridization may be varied during subsequent rounds of assembly. This results in a high degree of parallelism and synthesis of oligonucleotides with different sequences.

Automated or semi-automated systems may be used to introduce oligonucleotide hairpins, invading strands, and ligase to the surface of a substrate to create oligonucleotides with specific sequences of payload regions. Such systems may be computer controlled and selectively bring oligonucleotide hairpins encoding arbitrary information into contact with anchor strands on the substrate in a specific order. Doing so encodes a specific string of arbitrary information in the assembled oligonucleotide.

This Summary is provided to introduce a selection of concepts in a simplified form that are further described below in the Detailed Description. This Summary is not intended to identify key features or essential features of the claimed subject matter nor is it intended to be used to limit the scope of the claimed subject matter. The term “techniques,” for instance, may refer to system(s) and/or method(s) as permitted by the context described above and throughout the document.

### BRIEF DESCRIPTION OF THE DRAWINGS

The Detailed Description is set forth with reference to the accompanying figures. In the figures, the left-most digit(s) of a reference number identifies the figure in which the reference number first appears. The use of the same reference numbers in different figures indicates similar or identical items. The figures are schematic representations and items shown in the figures are not necessarily to scale.

FIGS. 1A and 1B are a time series illustrating the creation of oligonucleotides with specific sequences of payloads by sequential hybridization and ligation of oligonucleotide hairpins.

FIG. 2 is a time series illustrating the use of a microelectrode array to control the location of oligonucleotide hairpin hybridization.

FIG. 3 is a diagram illustrating example structures of oligonucleotide hairpins and invading strands used to encode two binary digits.

FIG. 4 is a flow diagram showing an illustrative process for assembling one or more oligonucleotides encoding specific arbitrary sequences from oligonucleotide hairpins.

FIG. 5 is an illustrative system for creating oligonucleotides by implementing techniques of this disclosure.

FIG. 6 is an illustrative computer architecture for implementing techniques of this disclosure.

### DETAILED DESCRIPTION

This disclosure provides techniques that use hybridization between overhanging ends of oligonucleotide hairpins and anchor strands to selectively assemble oligonucleotides that encode arbitrary information. The oligonucleotide hairpins are joined to anchor strands attached to a solid substrate. Thus, this provides a technique for solid-state oligonucleotide synthesis. The arbitrary information may be any type of information that can be encoded in a sequence of nucleotides. For example, the arbitrary information may be binary digits (e.g., 0 and 1). Oligonucleotides that encode binary digits may be used to encode digital information. Encoding schemes for representing binary digits with nucleotide sequences are known to those of ordinary skill in the art. Any other type of arbitrary information such as trits or ASCII characters may also be encoded.

The disclosed technique for assembling oligonucleotides uses the enzyme ligase and pre-synthesized oligonucleotides structures-oligonucleotide hairpins and invading strands. This is accomplished without the use of phosphoramidite chemical synthesis or template-independent DNA polymerase enzymatic synthesis. Thus, the chemical waste from phosphoramidite chemical synthesis and the challenges associated with regulating enzymatic synthesis are avoided. This disclosure provides a novel technique for generating oligonucleotides that encode arbitrary information using only pre-synthesized oligonucleotide structures and the enzyme ligase.

These techniques are readily adapted for automated or semiautomated systems such as microfluidic or laboratory robotics systems. The use of a microelectrode array allows for massively parallel creation of a large number of oligonucleotides with different sequences. This enables efficient encoding of a large amount of arbitrary information. One application for the techniques of this disclosure is a DNA data storage center. In a DNA data storage center, a large amount of digital information is encoded in oligonucleotides such as DNA. A DNA data storage center may continually write digital information through the synthesis of oligonucleotides. If done with the conventional phosphoramidite technique, such a large amount of de novo synthesis would generate significant amounts of hazardous organic waste.

Oligonucleotides, also referred to as polynucleotides, include both DNA, ribonucleic acid (RNA), and hybrids containing mixtures of DNA and RNA. DNA includes nucleotides with one of the four natural bases cytosine (C), guanine (G), adenine (A), or thymine (T) as well as unnatural bases, noncanonical bases, and modified bases. RNA includes nucleotides with one of the four natural bases cytosine, guanine, adenine, or uracil (U) as well as unnatural bases, noncanonical bases, and modified bases.

Detail of procedures and techniques not explicitly described or other processes disclosed in this application are understood to be performed using conventional molecular biology techniques and knowledge readily available to one of ordinary skill in the art. Specific procedures and techniques may be found in reference manuals such as, for example, Michael R. Green & Joseph Sambrook, Molecular Cloning: A Laboratory Manual, Cold Spring Harbor Laboratory Press, 4^{th} ed. (2012).

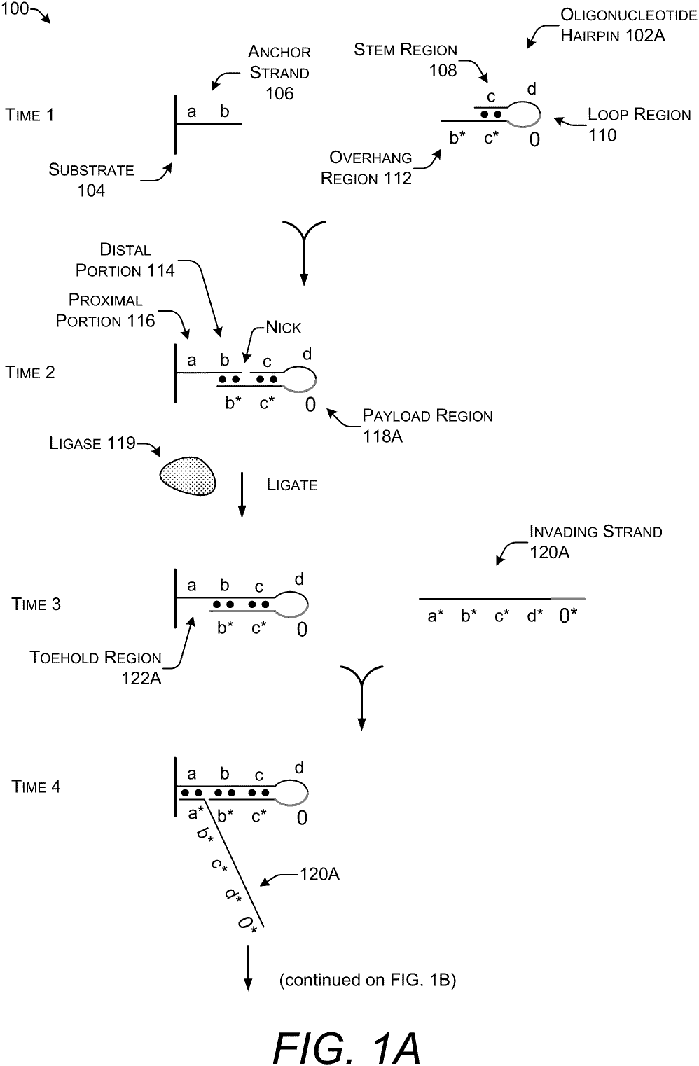

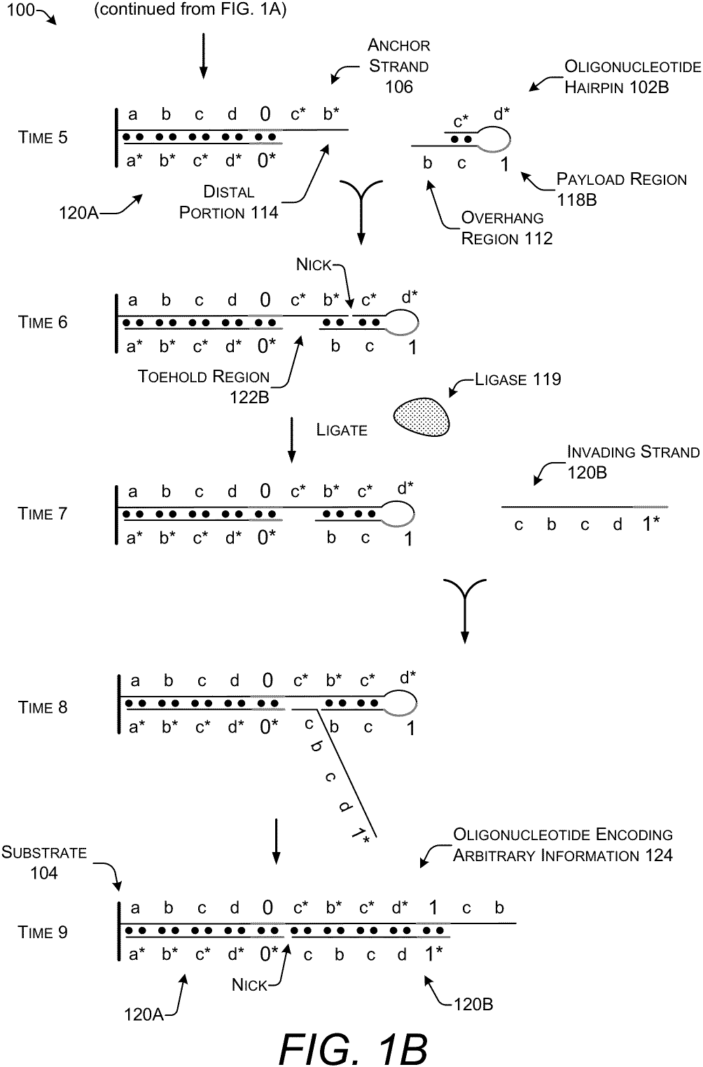

FIGS. 1A and 1B show a time series 100 that illustrates the creation of oligonucleotides with specific sequences of payloads by sequential hybridization and ligation of oligonucleotide hairpins 102. Although this time series 100 is shown as a sequence of discrete steps, any two or more of the steps may be combined unless expressly indicated otherwise.

Assembly of the oligonucleotides begins at Time 1 with a substrate 104 that is coated with anchor strands 106. The substrate 104 may be any type of substrate 104 suitable for solid-phase oligonucleotide synthesis. Persons of ordinary skill in the art can readily select an appropriate substrate for solid-phase oligonucleotide synthesis. The substrate 104 may be formed from a material such as glass, silicon, or plastic. In an implementation, the substrate 104 is a flat or substantially flat surface such as a silicon chip or glass slide. In an implementation, the substrate 104 is a bead or microsphere. The substrate 104 may also be implemented as magnetic nanoparticles one example of which are “TurboBeads®” available from TurboBeads LLC (Zürich, Switzerland). TurboBeads are described in Grass et al., Covalently Functionalized Cobalt Nanoparticles as a Platform for Magnetic Separations in Organic Synthesis, 46 Angew. Chem. Int. Ed. 4909 (2007).

The anchor strands 106 on the substrate 104 are single-stranded oligonucleotides. The oligonucleotides in the anchor strands 106 may be synthesized by conventional phosphoramidite synthesis, enzymatic synthesis, or any other technique. Each anchor strand 106 may be about 5-15 nucleotides long. For example, an anchor strand 106 may be about 9-11 nucleotides long. However, the anchor strands 106 may be longer or shorter. Although only a single anchor strand 106 is illustrated in FIG. 1, the substrate 104 may be coated with thousands or millions of individual anchor strands 106. In an implementation, all anchor strands 106 have the same sequence of nucleotides. There may also be other implementations in which there are multiple different anchor strands 106 with different sequences over at least part of their lengths.

The anchor strands 106 may be attached to the substrate 104 by any known technique for coating a solid substrate with oligonucleotides. Multiple techniques are known to those of ordinary skill in the art including techniques used to generate DNA microarrays such as spotting or printing with an inkjet-like printer, in situ synthetization, and bead arrays. For discussion of different microarray platforms that may be used to generate a substrate coated with oligonucleotides see Miller MB, Tang YW. Basic concepts of microarrays and potential applications in clinical microbiology. Clin Microbiol Rev. 2009 Oct;22(4):611-33.

The substrate 104 may, in some implementations, be functionalized to provide for attachment of the anchor strands 106. Examples include silane functionalization which covers a surface with organofunctional alkoxysilane molecules and agarose functionalization which covers a surface with a polysaccharide matrix. Linkers may be used to attach the anchor strands 106 to the surface of the substrate 104. Examples of linkers that may be used are provided in U.S. Pat. Pub. No. 2020/0199662 filed on Dec. 21, 2018, with the title “Selectively Controllable Cleavable Linkers.” Non-covalent attachment such as streptavidin-biotin interactions may also be used to attach the anchor strands 106 to the substrate 104. These and other techniques for attaching single-stranded oligonucleotides to a solid substrate are well known to those of ordinary skill in the art.

In this example time series 100, the anchor strand 106 is contacted with a first oligonucleotide hairpin 102A. The oligonucleotide hairpins 102 may be synthesized by conventional phosphoramidite synthesis, enzymatic synthesis, or any other technique such as cloning in bacteria. Oligonucleotide hairpins 102 include a stem region 108 and a loop region 110. In order to form a loop structure, the loop region 110 is typically at least 3 nucleotides long. The loop region 110 may be longer than 3 nucleotides. The stem region 108 stabilizes the hairpin structure and is typically 6 or more nucleotides. However, the stem region 108 may be shorter than 6 nucleotides. The stem region 108 is double-stranded and includes a first strand indicated by “c” that hybridizes to the second strand indicated by “c*”. The notation of n* indicates a sequence that hybridizes to or is complementary to n where n represents a single-stranded oligonucleotide sequence. Thus, a* hybridizes to sequence a, b* hybridizes to sequence b, and so forth. Sequences with less than full complementarity may hybridize to each other.

The oligonucleotide hairpins 102 of this disclosure additionally include an overhang region 112. The overhang region 112, illustrated as “b*”, is a region of single-stranded oligonucleotides that extends from one side of the stem region 108. The overhang region 112 hybridizes to a distal portion 114 of the anchor strand 106 illustrated as “b.” The overhang region 112 and the distal portion 114 may be the same length. For example, both may be at least 3 nucleotides or at least 4 nucleotides. The overhang region 112 and the distal portion 114 may be longer than 4 nucleotides.

Techniques for designing stable hairpin structures are well known to those of ordinary skill in the art. Oligonucleotide hairpins may be designed using software created for that purpose such as, but not limited to, NUPACK available from nupack.org. For a discussion of the NUPACK software see J. N. Zadeh, C. D. Steenberg, J. S. Bois, B. R. Wolfe, M. B. Pierce, A. R. Khan, R. M. Dirks, N. A. Pierce. NUPACK: analysis and design of nucleic acid systems. J Comput Chem, 32:170-173, 2011.

Time 2 illustrates hybridization between the overhang region 112 of an oligonucleotide hairpin 102 and the distal portion 114 of an anchor strand 106. Hybridization between two single-stranded oligonucleotides is represented as a series of black dots. The anchor strand 106 is designed so that a proximal portion 116, illustrated as “a”, remains as a single-stranded structure following hybridization of the oligonucleotide hairpin 102. The proximal portion 116 of the anchor strand 106 is those nucleotides of the anchor strand 106 that are closest to the substrate 104. In an implementation, the proximal portion 116 may be at least 6 nucleotides. For example, the proximal portion 116 may be 6 and 7 nucleotides. However, the proximal portion 116 may be shorter or longer.

The loop region 110 includes a payload region 118. For the first oligonucleotide hairpin 102A, the first payload region 118A encodes a binary digit represented in FIG. 1 as “0.” Hybridization between the first oligonucleotide hairpin 102A and the anchor strand 106 associates the arbitrary information represented by the payload region 118A with a specific location on the surface of the substrate 104.

The payload region 118A is at least one nucleotide long but may be much longer. The length of the payload region 118A depends upon the arbitrary information and the encoding scheme. For example, the nucleotide base G or C may be used to encode the binary digit 0 while the nucleotide base A or T may be used to encode the binary digit 1. A length of the payload region 118A can be 1-5 nucleotides. To provide a more robust encoding, more than one nucleotide may be used. For example, the nucleotide sequence ATAT may be used to encode 1 while the nucleotide sequence GCGC is used to encode 0. In an implementation, the length of the payload region 118A is 2-5 nucleotides. However, the payload region 118A may be longer than 5 nucleotides.

The loop region 110 may optionally include one or more additional nucleotides besides those in the payload region 118A. The additional nucleotides in the loop region 110 are represented by “d.” However, if the additionally nucleotides are not present, the “d” portion of the loop region 110 is omitted. If the length of the payload region 118 is less than 3 nucleotides, the additional nucleotides may be added so that the loop region 110 has sufficient length and flexibility to form a loop structure. Additional, non-payload nucleotides in the loop region 110 may be added to stabilize the structure of the oligonucleotide hairpin 102. In an implementation, the specific nucleotides may be determined by a software program such as NUPACK.

The overhang region 112 extends from one side of the stem region 108, the “overhang side,” such that when hybridized to the distal portion 114 of the anchor strand 106, the other side of the stem region 108, the “non-overhang side,” is positioned adjacent to the nucleotide on the end of the anchor strand 106. This is illustrated in FIG. 1 by “c” being positioned adjacent to “b.” Preferentially, the end of the non-overhang side of the oligonucleotide hairpin 102 is directly adjacent to the end of the anchor strand 106 such that there is no space for an additional nucleotide in between. This results in a nick in the backbone of the double-stranded oligonucleotide formed from b/b* and c/c*. In some implementations, there may be a one or two nucleotide gap between the end of the stem region 108 and the end of the anchor strand 106. However, ligation efficiency will decrease if the ends are not directly adjacent.

Time 3 shows a connection formed between the oligonucleotide hairpin 102 and the anchor strand 106 following contact with ligase 119. In some implementations, ligase 119 may be added together with the oligonucleotide hairpin 102. Nicks in an oligonucleotide backbone are closed by ligation. Techniques for performing ligation and closing of nicks in DNA and RNA are well-known to those of ordinary skill in the art. For example, techniques used to join DNA fragments in Golden Gate Assembly may be readily adapted for use in ligating the oligonucleotide hairpin 102 to the anchor strand 106.

Ligases for both DNA and RNA are known. DNA ligase is a specific enzyme that joins DNA strands together by catalyzing the formation of a phosphodiester bond. One specific type of DNA ligase that is frequently used in molecular biology is T4 DNA Ligase isolated from bacteriophage T4. T4 DNA ligase is most active at 37° C. RNA ligase (ATP) is an analogous enzyme that catalyzes the formation of phosphodiester bonds between ribonucleotides. One commercially available RNA ligase suitable for closing nicks is T4 RNA ligase 2. T4 RNA ligase 2 is also most active at 37° C. DNA and RNA ligases including appropriate ligase buffers are available from multiple commercial sources. For example, a ligase buffer may contain 50 mM Tris-HCl, 10 mM MgCl₂, 1 mM ATP, 10 mM DTT, with pH 7.5 at 25° C.

For optimal ligation efficiency with sticky ends, the optimal temperature for the enzyme is balanced with the melting temperature Tₘ of the sticky ends being ligated because the homologous pairing of the sticky ends may be disrupted by high temperatures. If any of the sticky ends in the double-stranded oligonucleotide structure shown at Time 5 would be disrupted at optimal temperatures for the selected ligase, a lower temperature may be used. Persons of ordinary skill in the art will understand how to calculate Tₘ for a given oligonucleotide structure and adjust the ligation temperature appropriately.

At Time 3, the structure formed from ligation of the oligonucleotide hairpin 102 to the anchor strand 106 is contacted with an invading strand 120. In one implementation, the oligonucleotide hairpin 102, the ligase 119, and invading strand 120 may be added together so the mixture of all three contacts the anchor strand 106. In another implementation, the ligase 119 and the invading strand 120 may be added together after addition of the oligonucleotide hairpin 102.

The proximal portion 116 of the anchor strand 106 that remains as a single-stranded structure provides a toehold region 122 for attachment of the invading strand 120. As used herein, the term “toehold” refers to a short (e.g., comprising 1-10 or at least 6 (e.g., 1, 2, 3, 4, 5, 6, 7, 8, 9, or 10 nucleotides)) single-stranded nucleic acid extension that is adjacent to a duplex and that accelerates the binding of a third oligonucleotide to one of the strands of the duplex and that displaces one strand of the original duplex. Thus, a “toehold” may provide a nucleation site of an invading strand 120 that is configured to initiate hybridization of a complementary nucleic acid sequence. Techniques for toehold mediated strand displacement are well known to those of ordinary skill in the art and discussed in D. Zhang and E. Winfree, Control of DNA Strand Displacement Kinetics Using Toehold Exchange. Am. Chem. Soc. 131:17303-17314, 2009.

This first toehold region 122A is the proximal portion 116 of the anchor strand 106. Thus, the first toehold region 122A has a length of preferentially 6 or 7 nucleotides. This length of a toehold region has been shown to provide favorable kinetics for strand displacement. The anchor strand 106 may, however, include additional nucleotides between the sequence that functions as the first toehold region 122A (i.e., “a”) and the substrate 104. Thus, the portion of the anchor strand 106 between the substrate 104 and the distal portion 114 may include multiple nucleotides adjacent to the substrate 104 that do not function as the toehold region 122A.

The first invading strand 120A hybridizes to the anchor strand 106 and to portions of the first oligonucleotide hairpin 102A other than the overhang region 112 and the overhang side of the stem region 108. This is illustrated in FIG. 1A by the first invading strand 120A hybridizing to c, d, and 0 of the first oligonucleotide hairpin 102A and not hybridizing to “c*” and “b*”.

Time 4 shows hybridization of the invading strand 120 before the displacement of the double-stranded structure formed from the oligonucleotide hairpin 102 and the anchor strand 106. One end of the invading strand 120, represented by “a*”, hybridizes to the toehold region 122. This creates an oligonucleotide complex formed from one looped strand and one single-stranded oligonucleotide. This initial endothermic step is rate limiting and can be tuned by varying the strength (length and sequence composition e.g., G-C or A-T rich strands) of the toehold region 122. Techniques for tuning the kinetics of strand displacement are known to those of ordinary skill in the art. The sequence of each invading strand 120 will be specific and will hybridize to portions of a specific type of oligonucleotide hairpin 102. Here, the first invading strand 120A hybridizes to portions of the first oligonucleotide hairpin 102A. The invading strand 120 opens the hairpin through branch migration.

This may be followed with a washing step to remove any free first oligonucleotide hairpins 102A or first invading strands 120A from the solution in contact with the substrate 104. Additionally or alternatively, one or more species of endonucleases that digest single-stranded oligonucleotides may be added to the solution in contact with the substrate 104. An endonuclease will digest invading strands 120 and the overhang region 112 of any oligonucleotide hairpins 102 to prevent hybridization that could interfere with the addition of additional oligonucleotide hairpins 102.

FIG. 1B begins with Time 5 illustrating hybridization of the first invading strand 120A and opening of the first oligonucleotide hairpin 102A. Displacement and opening of the stem region of the hairpin are energetically favored because the invading strand 120 forms a longer double-stranded structure once fully hybridized. In this example, the first invading strand 120A includes regions (i.e., d* and 0*) that hybridized to and form a double-stranded structure with nucleotides of the loop region 110 (i.e., d and 0). Allowing the nucleotides of the loop region 110 to form a double-stranded structure achieves a lower energy state than remaining in a hairpin confirmation.

After the opening of the first oligonucleotide hairpin 102A, the overhang side of the stem region (b* and c*) now becomes the end of the anchor strand 106. The former overhang region 112 of the first oligonucleotide hairpin 102A (b*) now takes the place of the distal portion 114 of the anchor strand 106. At Time 5, the first invading strand 120A may remain hybridized to the anchor strand 106. However, the first invading strand 120A is not covalently attached to the substrate 104. Alternatively, and not shown, the double-stranded structure may be denatured and the first invading strand 120A may be washed away.

The anchor strand 106, which now includes the nucleotides of the first oligonucleotide hairpin 102A, is contacted with a second oligonucleotide hairpin 102B. The second oligonucleotide hairpin 102B has a different sequence than the first oligonucleotide hairpin 102A. The second oligonucleotide hairpin 102B includes an overhang region 112, represented by “b”, that hybridizes to the distal portion 114 of the anchor strand 106. Thus, the overhang regions 112 of the first oligonucleotide hairpin 102A and the second oligonucleotide hairpin 102B hybridize to each other.

The second oligonucleotide hairpin 102B includes a second payload region 118B in the loop region 110. The second payload region 118B may encode the same or different arbitrary information than the first payload region 118A. In this example, the second payload region 118B includes a binary digit represented as “1.” If includes the same arbitrary information, then the sequence of the second payload region 118B will be the same as that of the first payload region 118A.

At Time 6 following hybridization of the second oligonucleotide hairpin 102B and the anchor strand 106, there is a second toehold region 122B. The second toehold region 122B exists because the first invading strand 120A does not extend to nucleotides in the first oligonucleotide hairpin 102A represented as “c*” and the overhang region 112 of the second oligonucleotide hairpin 102B hybridizes with only the distal portion 114 of the anchor strand 106. A length of the second toehold region 122B may be preferentially 6 or 7 nucleotides. However, it may also be a different length. The length of the second toehold region 122B is the same as the length of the stem region 108 of the first oligonucleotide that hairpin 1028. This is because the overhang side of the stem region 108 (i.e., “c*”) becomes the second toehold region 122B.

Following hybridization of the second oligonucleotide that hairpin 102B to the anchor strand 106, there is again a nick or gap between the end of the non-overhang side of the stem region (“c*”) and the nucleotide on the end of the anchor strand 106. The size of this nick and the positioning of second oligonucleotide hairpin 102B relative to the end of the anchor strand 106 is determined by alignment of the sequences in the overhang region 112 of the second oligonucleotide hairpin 102B (b) and the distal portion 114 of the anchor strand 106 (“b*”). The two nucleotides are preferably directly adjacent to each other so that the formation of a single phosphodiester bond will join the oligonucleotide background. However, in some implementations, the gap may be one or two nucleotides.

Ligase 119 is used to close the nick and join the backbone as shown in Time 7. As described above, the ligase 119 may be added at the same time as the second oligonucleotide hairpin 102B. The structure formed from closing the nick is contacted with a second invading strand 120B. The second invading strand 120B may, in some implementations, be added at the same time as the second oligonucleotide hairpin 102B and the ligase 119. Alternatively, the second invading strand 120B and the ligase 119 may be added together after addition of the second oligonucleotide hairpin 102B. The second invading strand 120B initially hybridizes to the single-stranded toehold region 122B as shown in Time 8. The second invading strand 120B hybridizes to the anchor strand 106 and to portions of the second oligonucleotide hairpin 102B other than the overhang region 112 and the overhang side of the stem region 108. This is illustrated in FIG. 1B by the second invading strand 120B hybridizing to “c*”, “d*”, and 1 of the second oligonucleotide hairpin 102B and not hybridizing to c and b.

Time 9 illustrates complete hybridization of the second invading strand 120B and opening of the second oligonucleotide that hairpin 102B. The portions of the second oligonucleotide hairpin 102B that was the overhang region 112 and the overhang side of the stem region 108 (i.e., “b” and “c”) now form the end of the anchor strand 106. Now the anchor strand 106 has been extended by the addition of two oligonucleotide hairpins 102. In this example, the oligonucleotide encoding arbitrary information 124 that is formed by this process encodes binary digit 0 followed by the binary digit 1. This process may be repeated with additional oligonucleotide hairpins 102 to create an oligonucleotide that encodes any sequence of arbitrary information such as a string of binary digits.

At Time 9 there is a nick between the ends of the first invading strand 120A and the second invading strand 120B. If both invading strands 120 remain hybridized to the growing anchor strand 106, a later round of ligation will close this nick. This will create a double-stranded oligonucleotide with only one strand is attached to the substrate 104.

Due to incorporation of the overhang region 112 and the stem region 108 of the oligonucleotide hairpins 102, the oligonucleotide assembled on the substrate 104 is mostly regions that do not encode arbitrary information. This is shown in FIG. 1 by nucleotides that do not encode information (i.e., the region “c*”, “b*”, “c*”, “d*”) between the payload regions 118 that encode 0 and 1.

This technique involves hybridization of oligonucleotide hairpins 102, anchor strands 106, and invading strands 120. The sequence of oligonucleotides or oligonucleotide regions that hybridize to each other may be complementary but it is understood that they need not be 100% complementary. As used herein, the terms “complementary” or “complementarity” are used in reference to oligonucleotides related by the base-pairing rules. “Complementary” or “complementarity” refers to the nucleotides of a nucleic acid sequence that can bind to another nucleic acid sequence through hydrogen bonds, e.g., nucleotides that are capable of base pairing, e.g., by Watson-Crick base pairing or other base pairing. Nucleotides that can form base pairs, e.g., that are complementary to one another, are the pairs: cytosine and guanine, thymine and adenine, adenine and uracil, and guanine and uracil. Complementarity may be “partial,” in which only some of the nucleic acids’ bases are matched according to the base-pairing rules. Or there may be “complete” or “total” complementarity between the nucleic acids. The degree of complementarity between oligonucleotides has significant effects on the efficiency and strength of hybridization between nucleic acid strands.

Oligonucleotide sequences that hybridize to each other may have, at least 50%, at least 60%, at least 70%, at least 80%, at least 90%, at least 95%, at least 99%, or 100% sequence complementarity. Percent complementarity between particular stretches of oligonucleotide sequences can be determined routinely using software such as the BLAST programs (basic local alignment search tools) and PowerBLAST programs known in the art (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

As used herein, the term “hybridization” is used in reference to the pairing of complementary nucleic acids. Hybridization and the strength of hybridization (e.g., the strength of the association between the nucleic acids) is influenced by such factors as the degree of complementary between the nucleic acids, stringency of the conditions involved, and the Tₘ of the formed hybrid. “Hybridization” methods involve the annealing of one nucleic acid to another, complementary nucleic acid, e.g., a nucleic acid having a complementary nucleotide sequence. The ability of two oligonucleotides comprising complementary sequences to find each other and anneal through base pairing interaction is a well-recognized phenomenon. The initial observations of the “hybridization” process by Marmur and Lane, Proc. Natl. Acad. Sci. USA 46: 453 (1960) and Doty et al., Proc. Natl. Acad. Sci. USA 46:461 (1960), have been followed by the refinement of this process into an essential tool of modern biology.

As used herein, the term “Tₘ” is used in reference to the “melting temperature.” The melting temperature is the temperature at which a population of double-stranded nucleic acid molecules becomes half dissociated into single strands. Several equations for calculating the Tₘ of nucleic acids are well known in the art. As indicated by standard references, a simple estimate of the Tₘ value may be calculated by the equation: Tₘ=81.5+0.41*(%G+C), when a nucleic acid is in an aqueous solution at 1 M NaCl (see, e.g., Anderson and Young, “Quantitative Filter Hybridization” in Nucleic Acid Hybridization (1985). Other references (e.g., Allawi and SantaLucia, Biochemistry 36: 10581-94 (1997),) include more sophisticated computations which account for structural, environmental, and sequence characteristics to calculate Tₘ.

Unless otherwise specified, hybridization, as used throughout this disclosure, refers to the capacity for hybridization between two single-stranded oligonucleotides or oligonucleotide segments at 21° C. in 1 × TAE buffer containing 40 mM TRIS base, 20 mM acetic acid, 1 mM ethylenediaminetetraacetic acid (EDTA), and 12.5 mM MgCl₂. Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and also in Sambrook, J. and Russell, W., Molecular Cloning: A Laboratory Manual, Third Edition, Cold Spring Harbor Laboratory Press, Cold Spring Harbor (2001). As is known to those of ordinary skill in the art, conditions of temperature and ionic strength determine the “stringency” of the hybridization.

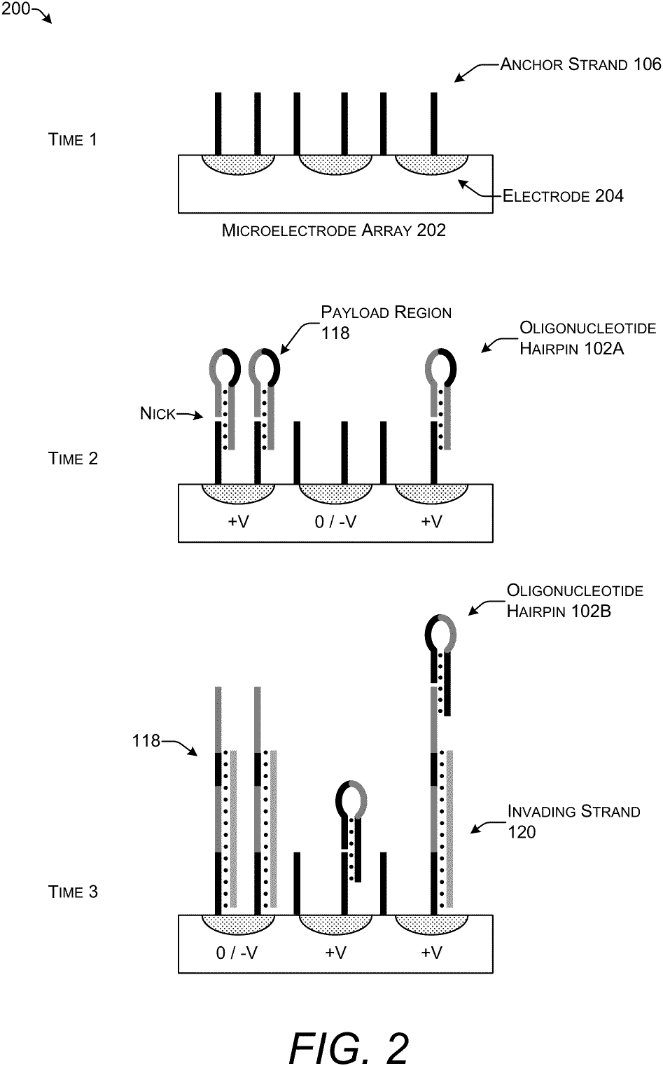

FIG. 2 shows a time series 200 that illustrates the use of a microelectrode array to control the location of oligonucleotide hairpin hybridization. The oligonucleotide hairpins 102, the anchor strands 106, and invading strands 120, as well as the general technique are the same as described above in FIG. 1. The techniques of this disclosure can be used on an inert flat substrate or bead to create multiple oligonucleotides with the same sequence. However, some applications, such as DNA data storage, benefit from the ability to create oligonucleotides with different sequences. A microelectrode array may be used to synthesize multiple different oligonucleotides with separate sequences in parallel on a single substrate. The different oligonucleotides could, for example, encode binary data corresponding to multiple different computer files.

The microelectrode array 202 shown in this time series 200 is illustrated with only three electrodes 204 but it is to be understood that the microelectrode array 202 may have many more electrodes 204. The microelectrode array may contain a large number of microelectrodes that make it possible to create many different oligonucleotides (e.g., 10,000, 60,000, 90,000, or more) on the surface of a single array. This high level of multiplexing is made possible by the microelectrode density which may be approximately 1000 microelectrodes/cm², 10,000 microelectrodes/cm², or a different density. Examples of suitable microelectrode arrays are provided in Bo Bi et al., Building Addressable Libraries: The Use of “Safety-Catch” Linkers on Microelectrode Arrays, 132 J. Am. Chem. Soc. 17,405 (2010), Nguyen et al., Scaling DNA Data Storage with Nanoscale Electrode Wells, 14:7 Science Advances (2021), and in U.S. Pat. Pub. No. 2020/0384434.

The microelectrode array 202 includes a plurality of electrodes 204 that can be independently activated to vary the charge across the surface of the microelectrode array 202. In one example implementation, the microelectrode array 202 is functionalized by spin coating with a 3 wt% solution of agarose in 1 × TBE buffer for 30 s at 1500 rpm. After coating, the microelectrode array 202 is baked at 50° C. for 1 h. This creates a surface with functional groups that can bind to the anchor strands 106. The anchor strands 106 may be synthesized directly onto the agarose coating using standard phosphoramidite reagents and methods.

At Time 1, the microelectrode array 202 is shown coated with anchor strands 106 represented as black bars. All of the anchor strands 106 on the microelectrode array 202 may have the same nucleotide sequence. Persons of ordinary skill in the art are aware of multiple ways to coat a microelectrode array 202 with single-stranded oligonucleotides. Techniques known to those of ordinary skill in the art for the generation of DNA microarrays may be adapted for this purpose. For example, the anchor strands 106 may be synthesized in situ using techniques such as those described in R. D. Egeland and E. M. Southern, Electrochemicallydirected synthesis of oligonucleotides for DNA microarray fabrication, Nucleic Acids Research, 2005 Vol. 33, No. 14. The surface of the microelectrode array 202 is covered with an aqueous solution that may be either an aqueous buffer solution or a mixed aqueous/organic solvent system. The aqueous solution in contact with the surface of the microelectrode array 202 is electrically conductive. The aqueous solution does not necessarily need buffering properties and may be a simple salt solution (e.g., 1 M NaCl).

Attachment of the anchor strands 106 to the surface of the microelectrode array 202 may not correlate in a one-to-one manner with the electrodes 204. Some electrodes 204 may have more than one anchor strand 106 attached. Some anchor strands 106 may be attached to a portion of the microelectrode array 202 that does not include an electrode 204. Some electrodes 204 may have no anchor strands 106 attached (not shown). However, all anchor strands 106 attached to the same electrode 204 will be exposed to the same electrochemical environment and generate the same oligonucleotides.

At Time 2, a first subset of the electrodes 204 is activated. As used herein, “activation” of an electrode 204 refers to causing the electrode 204 to have a positive charge relative to a reference electrode or to ground. In some implementations the positive charge may be about 3.3 V. Persons of ordinary skill in the art will be able to readily identify an appropriate voltage based on the design of the microelectrode array 202 and the solution in contact with the surface of the microelectrode array 202.

Electrically controlled hybridization or electro-assisted hybridization uses electrodes on the microelectrode array 202 to create positive charges that electrostatically attract negatively charged oligonucleotide hairpins 102. This attraction pulls the oligonucleotide hairpins 102 to only those electrodes 204 that currently have a positive charge and creates a higher localized concentration of oligonucleotide hairpins 102 in the solution near each positively charged electrode 204. This both creates site-selectivity, causing reactions to occur only those electrodes 204 that are activated with a positive charge, and concentrates oligonucleotide hairpins 102 in the region of the active electrodes, leading to a higher local concentration which can increase reaction kinetics. Electrodes 204 that are negatively charged or neutral (i.e., no charge) do not attract the oligonucleotide hairpins 102.

At Time 2, a first oligonucleotide hairpin 102A hybridizes to the anchor strands 106 in the proximity of the activated electrodes 204. Although the first oligonucleotide hairpin 102A is present in solution across the entire surface of the microelectrode array 202, it hybridizes in appreciable amounts only to those anchor strands 106 attached to activated electrodes. There may be some minimal amount of hybridization to anchor strands 106 that are not attached to positively charged electrodes 204, but this will generally be undetectable and not affect the sequences of the vast majority of oligonucleotides.

Hybridization results in a double-stranded oligonucleotide sequence as indicated by a series of black dots. A nick remains in between the end of the anchor strand 106 and the oligonucleotide hairpin 102 as described previously. This nick is closed by the activity of ligase. An invading strand 120 is added which opens the first oligonucleotide hairpins 120A and provides a single-stranded structure for hybridization of the second oligonucleotide that hairpin 102B. Typically, the current is maintained at the active electrodes 204 during the addition of the ligase and the invading strand 120. Keeping the electrodes 204 active prevents diffusion of oligonucleotide hairpins 102 across the entire surface of the microelectrode array 202 which may result in hybridization at unintended locations. Thus, the electrodes 204 are activated followed by addition of the oligonucleotide hairpins 102, the ligase, and the invading strands 120. Any two, or all three, of the oligonucleotide hairpins 102, the ligase, and the invading strands 120 may be added at the same time.

At Time 3, activation of a second subset of electrodes 204 illustrates how a second oligonucleotide hairpin 102B is directed to hybridize with a different set of anchor strands 106. Activation of the second subset of electrodes 204 is preceded by a washing step and/or endonuclease digestion to remove any of the first oligonucleotide hairpins 102A and invading strands 120 from the solution in contact with the surface of the microelectrode array 202. This process can be repeated by adding additional oligonucleotide hairpins 102 to the anchor strands 106.

The oligonucleotide hairpins 102 that are available in solution to hybridize with the anchor strands 106 may be changed during each round of assembly as illustrated by the addition of the first oligonucleotide hairpin 102A at Time 2 and the second oligonucleotide hairpin 102B at Time 3. This controls “what” (i.e., the payload region 118) is added to the anchor strands 106 attached to the microelectrode array 202. The selection of which electrodes 204 are activated controls “where” addition occurs. By varying the available oligonucleotide hairpins 102 and the activated electrodes 204, multiple oligonucleotides each with a different sequence of payload regions 118 are assembled in parallel on the microelectrode array 202.

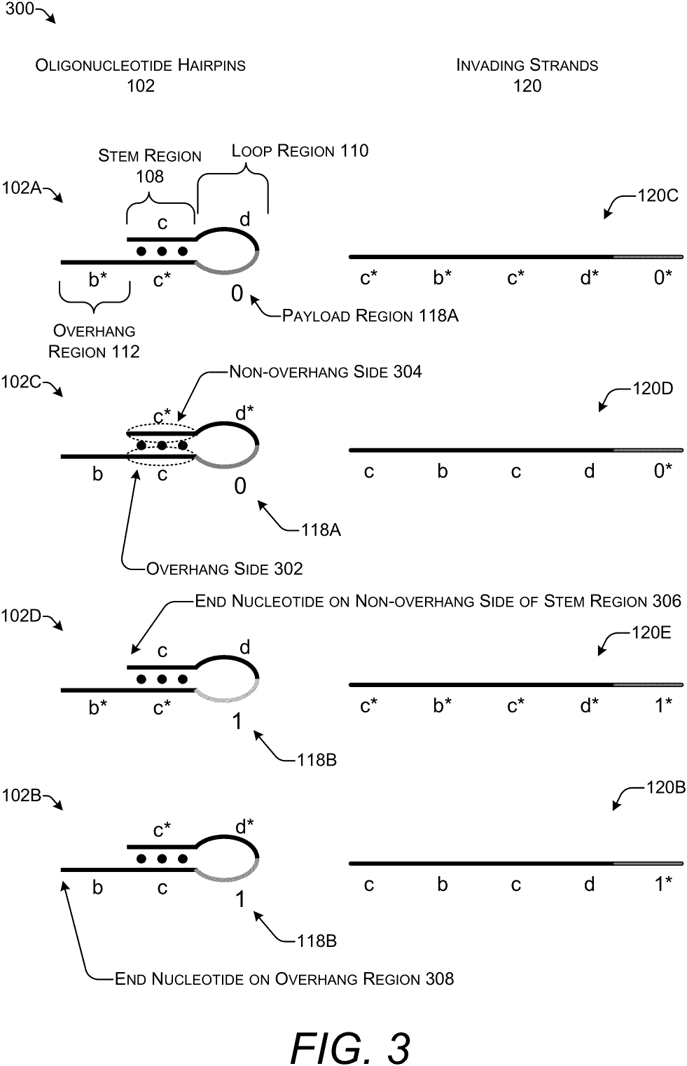

FIG. 3 is a diagram 300 illustrating example structures of oligonucleotide hairpins and invading strands used to create oligonucleotides that encode two binary digits. This diagram 300 shows the oligonucleotide components necessary to encode any arbitrary sequence of binary digits. There are two versions of oligonucleotide hairpins, 102A and 102C, that encode a first bit value which in this example is 0. There are also two versions of oligonucleotide hairpins, 102D and 102B, that encode a second bit value which in this example is 1. There is a corresponding invading strand 120B-E for each oligonucleotide hairpin 102A-D. Thus, with these eight oligonucleotide structures, the techniques of this disclosure can be used to create oligonucleotides encoding any arbitrary sequence of 0 and 1.

In this example, there are only two different payload regions 118A and 118B. The first payload region 118A encodes a first bit value 0. The second payload region 118B encodes a second bit value 1. A payload region 118 may be any length of one or more nucleotides. In one implementation, a length of the first payload region 118A and the second payload region 118B are each independently 2-5 nucleotides. In this example shown in FIG. 3, the payload regions 118A and 118B encode bit values, but a payload region 118 may encode any arbitrary information.

The sequence of a payload region 118 may use multiple nucleotides to encode a bit value such as CTA = 1 and ACG = 0. The payload region 118 may encode trits, letters of the English alphabet, or any other arbitrary information. The number of different variations of the payload region 118 will depend on the number of different pieces of arbitrary information that are encoded (e.g., two different payload regions for encoding bits, 26 different payload regions for encoding letters of the English alphabet, etc.). The number of oligonucleotide hairpins 102 and invading strands 120 necessary to encode all the different pieces of arbitrary information will increase correspondingly.

As discussed previously, each oligonucleotide hairpin 102 includes a stem region 108, a loop region 110, an overhang region 112, and a payload region 118. To provide for sufficiently strong hybridization to the anchor strand 106, in an implementation, a length of the overhang region 112 for each of the oligonucleotide hairpins 102A-D is independently at least 3 nucleotides.

The stem region 108 is a double-stranded structure and the side of the stem region 108 that is the same as the overhang region 112 is referred to as the overhang side 302. The other side of the stem region 108 that is not adjacent to the overhang region 112 is referred to as the non-overhang side 304. Recall from FIG. 1 that it is the non-overhang side 304 of the stem region 108 which becomes a toehold region 122 for hybridization of an invading strand 120. To provide a favorable speed of the strand displacement reaction, the toehold region 122 is preferably at least 6 nucleotides. Accordingly, in an implementation, the non-overhang side 304 of the stem region 108 for each of the oligonucleotide hairpins 102A-D are each independently at least 6 nucleotides. The overhang side 302 of the stem region 108 will have the same length as the non-overhang side 304.

The total length of an oligonucleotide hairpin 102 is typically at least 18 nucleotides. For example, an oligonucleotide hairpin 102 with an overhang region 112 containing 3 nucleotides, a stem region 108 that is 6 nucleotides long on each side, and a loop region 110 that is 3 nucleotides long will have a total length of 18 nucleotides. As an additional example, if the overhang region 112 is 4 nucleotides long, each side of the stem region 108 is 7 nucleotides, and a loop region 110 contains 5 nucleotides then the total length of the oligonucleotide hairpin 102 will be 23 nucleotides. Any of the overhang region 112, the stem region 108, and the loop region 110 may be longer than the example lengths provided above. Thus, there is no absolute upper limit on the length of an oligonucleotide hairpin 102. However, there is additional cost and effort associated with generating longer oligonucleotide sequences, so the shortest functional sequences may generally be preferred. Accordingly, in some implementations, the total length of an oligonucleotide hairpin 102 may be about 18-25 nucleotides.

The sequence of the distal portion 114 of the anchor strand 106 will change depending on the sequence of the last oligonucleotide hairpin 102 added. In this example, with only two different payload regions 118A and 118B, there are two possibilities for the sequence at the end of the anchor strand 106. One possibility is represented by the sequence “b” the other is the sequence “b*”. Accordingly, there are two different versions of the overhang region 112. The oligonucleotide hairpins 102A and 102D have an overhang region 112 with the sequence “b*” that will hybridize to the sequence “b”. The oligonucleotide hairpins 102B and 102C have an overhang region 112 with the sequence “b” will hybridize to the sequence “b*”. Thus, two different oligonucleotide hairpin 102 sequences are used to encode each bit value resulting in a total of four different oligonucleotide hairpins 102A-D.

The two versions of each oligonucleotide hairpin 102 can be thought of as a “positive version” and “negative version” for each bit value. In this example, the sequence of the oligonucleotide hairpin 102 other than the payload region 118 is the same for both “positive versions” and both “negative versions.” Thus, the sequence of oligonucleotide hairpin 102A and 102D are the same other than the payload regions 118A and 118B. Similarly, the sequence of oligonucleotide hairpin 102B and 102C are the same other than the payload regions 118A and 118B.

There is a similar relationship with the different versions of the invading strand 120. Each invading strand 120 hybridizes to all portions of an oligonucleotide hairpin 102 other than that overhang region 112 and the overhang side 302 of the stem region 108. Thus, each invading strand 120 hybridizes to the non-overhang side 304 of the stem region 108 and the loop region 110 (including the payload region 118) of the corresponding oligonucleotide hairpin 102. Taking oligonucleotide hairpin 102A and invading strand 120C as an example, the first c* and b* regions of the invading strand 120C will hybridize to the anchor strand 106 as shown in FIG. 1. The next portions of the invading strand 120C-c*, d*, and 0*- will hybridize to the non-overhang side 304 of the stem region 108 and the loop region 110 of the oligonucleotide hairpin 102A.

As discussed above, the portion of the loop region 110 that is not the payload region 118 (i.e., “d” or “d*”) may be omitted from oligonucleotide hairpins 102 in which case the corresponding region (i.e., “d*” or “d”) will also be omitted from the invading strand 120. However, in some implementations the loop region 110 of the oligonucleotide hairpins 102A-D includes at least one nucleotide that is not part of the payload region 118 (i.e., there is at least one nucleotide in the d/d* portion of the loop region 110). Nucleotides may be added to the loop region 110 to create a longer loop if, for example, the length of the payload region 118 is less than 3 nucleotides.

The total length of an invading strand 120 is the same as the corresponding oligonucleotide hairpin 102. Thus, invading strand 120 typically have a length of at least 18 nucleotides but may be longer such as 19, 20, 21, 22, 23, 24, 25, or more nucleotides.

Thus, oligonucleotide hairpin 102A is a first oligonucleotide hairpin comprising a first loop region that contains a first payload region 118A encoding the first arbitrary information (e.g., 0), a first stem region, and a first overhang region. Oligonucleotide hairpin 102C is a second oligonucleotide hairpin comprising a second loop region that contains the first payload region 118A encoding the first arbitrary information (e.g., 0), a second stem region, and a second overhang region that hybridizes to the first overhang region. Note that the sticky ends of oligonucleotide hairpin 102A and 102C will hybridize to each other if they are present in solution at the same time. However, each of the different species of oligonucleotide hairpins 102 is added separately and typically there is a washing step before the addition of the next species of oligonucleotide hairpin 102 so that the oligonucleotide hairpins 102 will not have an opportunity to hybridize to each other when used with the techniques of this disclosure.

Oligonucleotide hairpin 102D is a third oligonucleotide hairpin comprising a third loop region that contains a second payload region 118B encoding the second arbitrary information (e.g., 1), the first stem region, and the first overhang region that hybridizes to the second overhang region. Oligonucleotide hairpin 102B is a fourth oligonucleotide hairpin comprising a fourth loop region that contains the second payload region 118B encoding the second arbitrary information (e.g., 1), the second stem region, and the second overhang region that hybridizes to the first overhang region.

The invading strand 120C is a first invading strand that hybridizes to portions of the first oligonucleotide hairpin 102A other than to the first overhang region and an overhang side of the first stem region. The invading strand 120D is a second invading strand that hybridizes to portions of the second oligonucleotide hairpin 102C other than to the second overhang region and an overhang side of the second stem region. The invading strand 120E is a third invading strand that hybridizes to portions of the third oligonucleotide hairpin 102D other than to the first overhang region and the overhang side of the first stem region. The invading strand 120B is a fourth invading strand that hybridizes to portions of the fourth oligonucleotide hairpin 102B other than to the second overhang region and the overhang side of the second stem region.

The first invading strand 120C and the second invading strand 120D (as well as the third invading strand 120D and fourth invading strand 120B) can hybridize to each other along their length except for the portions corresponding to the payload region 118 (i.e., “0*”). However, multiple different invading strands 120 will not be present in solution together. There will be one or more washing steps between the addition of each different species of invading strand 120.

The sequences of the overhang regions 112 and the length of those regions in the respective varieties of oligonucleotide hairpins 102 may be such that when a first oligonucleotide hairpin (e.g., 102D) hybridizes to the opened end of a second oligonucleotide hairpin (e.g., 102B) the two ends are positioned as shown at Time 6 in FIG. 1B. This results in a single nick in the oligonucleotide backbone that may be readily closed by ligase. Stated differently, a first oligonucleotide hairpin (e.g., 102D) and a second nucleotide hairpin (e.g., 102B) have nucleotide sequences such that an end nucleotide on a non-overhang side of the first stem region 306 of the first oligonucleotide is directly adjacent to an end nucleotide on the second overhang region 308 of the second oligonucleotide when the first overhang region of the first oligonucleotide hairpin is hybridized to the second overhang region of the second oligonucleotide hairpin. As used in this context, directly adjacent refers to the 5′-end of a first nucleotide being positioned sufficiently close to the 3′-end of a second nucleotide such that there is not sufficient space for another nucleotide to be inserted between the two.

Displacement of the hairpins of oligonucleotide hairpins 102 that hybridize to the original anchor strand 106 as shown in FIG. 1A may use an invading strand 120A that is different than any of the invading strand 120B-E shown in FIG. 3. The initial invading strand 120A in FIG. 1A hybridizes to a toehold region 122A created by the proximal portion 116 of the anchor strand 106. This proximal portion 116 of the original anchor strand 106 may have a sequence that is different than any of the portions of the oligonucleotide hairpins 102. In FIG. 1A this proximal portion 116 is represented by “a” and the corresponding invading strand 120A includes the sequence represented by “a*”. However, to reduce the number of different invading strands needed, is also possible that the proximal portion 116 of the original anchor strand 106 is created with the sequence that is found in the stem region 108 of the oligonucleotide hairpins 102. Thus, in FIG. 1A instead of the sequence represented by “a”, the proximal portion 116 of the anchor strand 106 can have a sequence represented by “c”. Thus, the proximal portion 116 of the anchor strand 106 will in this example implementation have the same sequence as the non-overhang side 304 of the stem region 108 of oligonucleotide hairpins 102A and 102D.

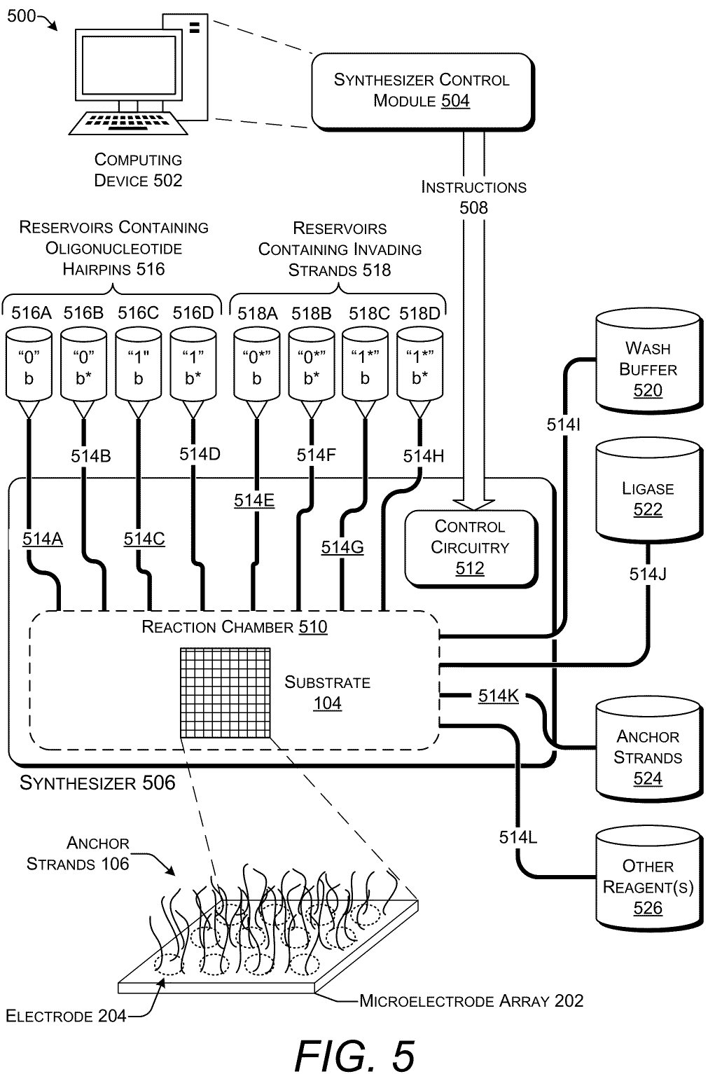

Each of the eight different oligonucleotide structures shown in diagram 300 (i.e., the first oligonucleotide hairpin 102A, the second oligonucleotide hairpin 102C, the third oligonucleotide hairpin 102D, the fourth oligonucleotide hairpin 102B, the first invading strand 120C, the second invading strand 120D, the third invading strand 120E, and the fourth invading strand 120B) may be present in different containers. Thus, they may be prepared as a set of reagents or a collection of oligonucleotide structures in which each species is stored in a different container. Any container suitable for the storage of oligonucleotides may be used. The container may contain an appropriate buffer for storing DNA or RNA. Such buffers are well known to persons or of ordinary skill in the art. For example, eight different vials, Eppendorf tubes, etc. may be provided in which each contains a large number of copies of each oligonucleotide species. In one implementation, the containers may be reservoirs used to supply reagents to a system for automated oligonucleotide assembly as shown in FIG. 5. If the payload regions 118 are used to encode more than two different types of arbitrary information, there will be a correspondingly larger number of unique oligonucleotide species and a larger number of containers necessary to separately hold each species.

Each of the oligonucleotide hairpins 102A-D and the invading strands 120C-E may be synthesized in advance and stored in separate containers as described above. Synthesis may be performed by any technique suitable for generating oligonucleotides with specific sequences such as conventional phosphoramidite synthesis. Although this disclosure provides techniques for assembling oligonucleotides from oligonucleotide hairpins 102 using only the enzyme ligase, if the oligonucleotide hairpins 102 and invading strands 120 themselves are created by phosphoramidite synthesis there is no net reduction in the use of hazardous chemicals such as acetonitrile. Rather it shifts the acetonitrile use and associated waste disposal from the site of oligonucleotide assembly to the site of oligonucleotide hairpin 102 and invading strand 120 synthesis.

There are techniques to manufacture many copies of an oligonucleotide sequence without phosphoramidite synthesis by cloning the sequence in bacteria. Techniques that use bacterial cloning to make multiple copies of a gene are well known to those of ordinary skill in the art. See Cohen SN, Chang AC, Boyer HW, Helling RB. Construction of biologically functional bacterial plasmids in vitro. Proc Natl Acad Sci USA. 1973 Nov; 70(11). The oligonucleotide sequences that form the oligonucleotide hairpins 102 and the invading strands 120 may be added to and manufactured in bacteria in the same manner as the DNA of a gene.

For example, oligonucleotide sequences corresponding to each of the eight oligonucleotide species (i.e., four oligonucleotide hairpins 102A-D and four invading strands 120B-E) may be inserted into plasmids and grown in E. coli. The specific oligonucleotide sequences may then be cut from the plasmids and purified using conventional techniques. One technique for producing single-stranded DNA that forms hairpins in E. coli is described in Ducani et al., Enzymatic production of ‘monoclonal stoichiometric’ single-stranded DNA oligonucleotides, Nature Methods, 10:7, 2013. Accordingly, in some implementations, the oligonucleotide hairpins 102 and the invading strands 120 are created by cloning in bacteria. The purified products of the bacteria cloning may then be stored in separate containers as described above.

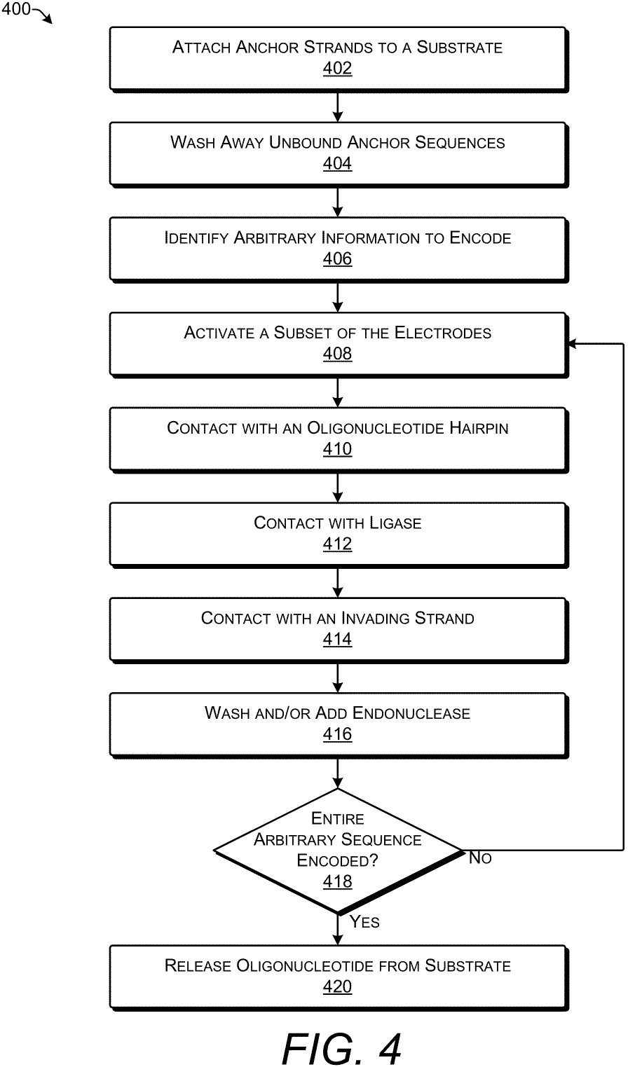

FIG. 4 shows process 400 for assembling one or more oligonucleotides encoding specific arbitrary sequences from oligonucleotide hairpins. For ease of understanding, the process 400 is delineated as separate operations represented as independent blocks. However, unless otherwise contradicted by context, any number of the described process blocks may be combined to implement the process or an alternate process. Moreover, it is also possible that one or more of the provided operations is modified or omitted.

At operation 402, anchor strands are attached to a substrate. The substrate may be any type of substrate suitable for solid-phase synthesis of oligonucleotides such as silicon, glass, or plastic. The substrate may be a generally flat surface or the substrate may be a bead. In an implementation, the substrate may be a microelectrode array as described above. The anchor strands may be attached to the substrate by any conventional technique for attaching oligonucleotide sequences to a solid substrate. For example, the surface of the substrate may be coated with linker molecules that in turn attach to an end of the anchor strands. As a further example, the surface of the substrate may be functionalized through silanization or coating with agarose. This creates a substrate that is coated with a plurality of anchor strands.

At operation 404, unbound anchor strands are washed away. This removes any anchor strands that are not attached to the microelectrode array. This washing step may be performed with water or an aqueous wash buffer. In some implementations, operations 402 and 404 may be omitted. Thus, process 400 may begin with a substrate that has been pre-coated with anchor strands.

At operation 406, arbitrary information to encode in an oligonucleotide is identified. Oligonucleotides created by the techniques of this disclosure may encode any type of arbitrary information including, but not limited to, a string of binary digits. For example, the arbitrary information may be identified by determining a string of binary digits that encodes digital information such as a computer file. The arbitrary information may be identified and stored in a computer system that is used to control an automated system for generating oligonucleotides. If multiple oligonucleotides are being assembled in parallel, such as by use of a microelectrode array, multiple different sequences of arbitrary information can be identified at operation 406.

At operation 408, if the substrate is a microelectrode array, a subset of electrodes in the microelectrode array is activated. If the substrate is an inert substrate and not a microelectrode array, operation 408 will be omitted. The subset of electrodes includes at least one electrode. Activation of the electrode(s) applies a positive charge which electrostatically attracts negatively charged oligonucleotides to a specific location on the surface of the substrate. The specific location may be varied during each round of oligonucleotide hairpin addition by activating a different subset of electrodes. Oligonucleotide hairpins present in solution are attracted to and hybridize with a first subset of anchor strands attached to the activated electrodes. The electrodes may remain activated during the following operations 410-414.

In one implementation, a voltage of +3.3 V may be applied for three cycles of 60 s with 10 s at 0 V between each cycle. Without being bound by theory, this length and duration of activation may provide the oligonucleotide hairpins with sufficient time to migrate to the electrodes and hybridize to the anchor strands. Specific voltages and timings of the electrode activation will be varied based on the design of the microelectrode array and the concentration of oligonucleotide hairpins in solution. Persons of ordinary skill in the art will be able to readily identify appropriate modifications to the strength and timing of electrode activation.

At operation 410, an anchor strand attached to the substrate is contacted with an oligonucleotide hairpin under conditions such that an overhang region of the oligonucleotide hairpin hybridizes to a distal portion of the anchor strand. The condition suitable for hybridization of two single-stranded oligonucleotide sequences may be the conditions described above or conditions known to persons of ordinary skill in the art. As discussed above, hybridization does not require fully complementary sequences but only that the strength of attachment between the oligonucleotide hairpin and the anchor strands is sufficient to hold the oligonucleotide hairpin in place until the subsequent ligation step.

The oligonucleotide hairpin includes a payload region that encodes arbitrary information within the loop region of the oligonucleotide hairpin. Hybridization between the oligonucleotide hairpin and the anchor strand leaves the proximal portion of the anchor strand adjacent to the substrate available as a single-stranded toehold region to hybridize with an invading strand.

At operation 412, the oligonucleotide (i.e., the oligonucleotide hairpin hybridized to the anchor strand) is contacted with ligase under conditions such that the ligase catalyzes the formation of a phosphodiester bond between the oligonucleotide hairpin and the anchor strand. In an implementation, operation 410 at operation 412 may be combined so that the oligonucleotide hairpins and the ligase are added at the same time. The use of ligase and suitable reaction conditions for joining the 5′-end and 3′-end of two oligonucleotides are well known to those of ordinary skill in the art. The ligase is added in an appropriate buffer such as a ligase buffer that contains ATP. After addition of the ligase, the oligonucleotide hairpin is covalently attached to the end of the anchor strand. The hairpin structure remains. Following addition of the ligase, any unbound oligonucleotide hairpins, as well as excess ligase, may be washed away with a wash buffer. Thus, only those oligonucleotide hairpins that have been ligated to an anchor strand remain.

At operation 414, the oligonucleotide is contacted with an invading strand under conditions such that the invading strand hybridizes to the toehold region of the anchor strand, the distal portion of the anchor strand, and a portion of the oligonucleotide hairpin including the payload region. Through toehold mediated strand displacement, the invading strand opens the hairpin structure of the oligonucleotide hairpin. In one implementation, operation 412 and operation 414 may be combined so that the ligase and the invading strands are added at the same time. In one implementation, operation 410, operation 412, and operation 414 may be combined so that the oligonucleotide hairpins, the ligase, and the invading strands are all added together.

The opening of the oligonucleotide hairpin creates a new single-stranded end to the anchor strand. This new end of the anchor strand is the overhang region and the overhang side of the stem region of the oligonucleotide hairpin. The invading strand remains hybridized to the remainder of the oligonucleotide hairpin and a portion of the anchor strand. The invading strand may be allowed to remain hybridized to the anchor strand. Alternatively, the double-stranded structure may be denatured and that invading strand may be washed away. Contacting the oligonucleotide with the invading strand may be followed by a wash step that removes any invading strands remaining in solution.

At operation 416, the surface of the microelectrode array or inert substrate is washed with a wash buffer to remove oligonucleotide species in solution. Additionally or alternatively, oligonucleotide hairpins and invading strands that remain in solution may be degraded by addition of an endonuclease that digests single-stranded oligonucleotides. This washing/endonuclease step is performed while the subset of electrodes are still activated. Thus, the electrodes are turned off only after the solution covering the microelectrode array is cleared of any oligonucleotides that could potentially hybridize with the anchor strands.

At operation 418, it is determined if the entire sequence of arbitrary information is encoded in the oligonucleotide. This determination can be performed by comparing the arbitrary information contained in the payload regions of the oligonucleotide hairpins added thus far to the entire sequence of arbitrary information. This determination may be made, for example, by a computer system controlling the automated assembly of the oligonucleotides.

Operations 408-416 are repeated adding additional oligonucleotide hairpins that each include an additional payload region that encodes additional arbitrary information. The additional arbitrary information may be the same or different than the arbitrary information added by the previous oligonucleotide hairpin. For example, a first bit “0” may be added followed by a second bit that is also “0.” The order of the oligonucleotide hairpins added is based on the order of the arbitrary information such as the order of 0′s and 1′s in a binary string. This process is repeated until the desired sequence of arbitrary information is represented in the oligonucleotide. During each round of addition, the overhang region of an oligonucleotide hairpin hybridizes to the nucleotides at the end of the anchor strand that were the overhang region of the previously added oligonucleotide hairpin.

If the arbitrary sequence has not been fully assembled, then process 400 proceeds along the “no” path and returns to operation 408 (or operation 410 if a microelectrode array is not used) where a subsequent oligonucleotide hairpin is introduced. As mentioned above, the subset of electrodes that are activated on the microelectrode array may be altered during each round of addition. Repeated cycles of adding oligonucleotide hairpins and activating selected subsets of electrodes enable the parallel creation of multiple different oligonucleotides with specified sequences of the arbitrary information. Thus, with this technique is possible to assemble at least a first oligonucleotide encoding first arbitrary information at a first location on the substrate and a second oligonucleotide encoding second arbitrary information at a second location on the substrate. If it is determined that the entire string (or the entirety of multiple strings) of arbitrary information such as a binary string is encoded in the oligonucleotide, process 400 proceeds along the “yes” path to operation 420.

At operation 420, the oligonucleotide created by repeated addition of oligonucleotide hairpins is released from the substrate. The oligonucleotides are released from the substrate by cleaving the connection between the base of the anchor strand and the substrate. The specific techniques for doing so will depend on how the anchor strand is attached to the substrate. If the anchor strand is attached to the substrate by a linker, the linker may be cleaved using a technique suitable for the chemistry of the particular linker. Numerous techniques for separating oligonucleotides bound to a solid substrate are known to those of ordinary skill in the art. For example, techniques used in the field of DNA micro arrays may be adapted for this purpose. All oligonucleotides attached to the surface of the substrate may be released in a single operation.

If the invading strands are not denatured during process 400, they will remain hybridized to the oligonucleotide that is attached to the substrate. Subsequent rounds of ligation will covalently attach each of these invading strands to each other. This will result in the formation of a double-stranded oligonucleotide. Only one strand of the double-stranded oligonucleotide, the strand that includes the original anchor strand, is attached to the substrate. Cleaving of that connection between the anchor strand and the substrate will release a double-stranded structure formed from the oligonucleotide hairpins and the invading strands. This double-stranded structure may be released as a double-stranded oligonucleotide or it may be denatured so there are two single-stranded oligonucleotides.

Denaturing may be performed by any suitable technique including, but not limited to, heating above the Tₘ of the assembled double-stranded oligonucleotide, adding sodium hydroxide, and increasing the salt concentration. These and other techniques for denaturing double-stranded oligonucleotides are well-known to those of ordinary skill in the art.

Following separation from the substrate, the oligonucleotide may be processed further such as, for example, by amplification with PCR. The PCR product may be stored for short or long term. The arbitrary information encoded in the oligonucleotide may be later obtained by sequencing the oligonucleotide and/or PCR amplification products.

FIG. 5 shows an illustrative system 500 that may include a computing device 502 with a synthesizer control module 504 that is communicatively connected to a synthesizer 506. The synthesizer control module 504 may provide instructions 508 that control the operation of the synthesizer 506. The instructions may cause the synthesizer 506 to create oligonucleotides with specific sequences and/or that encode specific information. The computing device 502 may be implemented as any type of conventional computing device such as a desktop computer, a laptop computer, a server, a hand-held device, or the like. In an implementation, the computing device 502 may be a part of the synthesizer 506 rather than a separate device.

The synthesizer 506 is a device that selectively assembles oligonucleotide hairpins through hybridization to anchor strands 106 followed by the opening of the hairpins with invading strands. Synthesis is performed on a substrate 104 coated with a plurality of anchor strands 106. The substrate may be implemented as a microelectrode array 202. The substrate 104 is located within a reaction chamber 510 or container capable of maintaining an aqueous or predominantly aqueous environment in contact with the surface of the substrate 104. Thus, the reaction chamber 510 is in fluid contact with the substrate 104. The synthesizer 506 may include a heater to control the temperature of the aqueous solution in the reaction chamber 510. The substrate 104, whether implemented as a microelectrode array 202 or not, may be created in advance and placed within the reaction chamber 510.

Control circuitry 512 may control the operation of the synthesizer 506. The control circuitry 512 may be implemented as any type of circuitry suitable for controlling hardware devices such as a printed circuit board, microcontroller, programmable logic controller (PLC), or the like. The control circuitry 512 receives the instructions 508 provided by the synthesizer control module 504. Instructions 508 may indicate the order of payload regions that are to be assembled at individual electrodes 204 on the microelectrode array 202.

The control circuitry 512 may also be configured to selectively activate individual electrodes 204 in the microelectrode array 202 with a voltage sufficient to attract oligonucleotides to the substrate. If the synthesizer 506 is implemented with a microelectrode array 202, the control circuitry 512 can be configured to cause, through selective activation of individual electrodes 204 in the microelectrode array 202, the system 500 to assemble at least a first oligonucleotide encoding a first sequence of arbitrary information at a first location on the substrate 104 and a second oligonucleotide encoding a second sequence of arbitrary information at a second location on the substrate 104. In typical applications, thousands or millions of different oligonucleotides can be assembled on the surface of the microelectrode array 202.

The control circuitry 512 may also be able to activate fluid delivery pathways 514 that control movement of fluids throughout the synthesizer 506 including into the reaction chamber 510. The fluid delivery pathways 514 may be implemented by tubes and pumps, microfluidics, laboratory robotics, or other techniques known to those of ordinary skill in the art for moving fluids.

For example, microfluidic technology facilitates the automation of chemical and biological protocols. These devices manipulate small quantities of liquid at smaller scales and with higher precision than humans. Digital microfluidic (DMF) technology is one type of flexible microfluidic technology. DMF devices manipulate individual droplets of liquids on a grid of electrodes, taking advantage of a phenomenon called electrowetting on dielectric. Activating electrodes in certain patterns can move, mix, or split droplets anywhere on the chip. Microfluidics also includes full-stack microfluidics which are programmable systems that allow the unrestricted combination of computation and fluidics. Examples of microfluidic technology may be found in Willsey et al., Puddle: A dynamic, error-correcting, full-stack microfluidics platform, Aplos′ 19, April 13-17, 183 (2019).

In an implementation, the synthesizer 506 may include multiple reservoirs containing oligonucleotides and other reagents used by the synthesizer 506. The synthesizer 506 may include a first set of reservoirs containing oligonucleotide hairpins 516. The oligonucleotide hairpins may be any of the oligonucleotide hairpins described in this disclosure such as the oligonucleotide hairpins 102A-D shown in FIG. 3. Each unique species of oligonucleotide hairpin may be stored in a separate reservoir 516A-D. Thus, the synthesizer 506 may include a first reservoir 516A containing the first oligonucleotide hairpin, a second reservoir 516B containing a second oligonucleotide hairpin, a third reservoir 516C containing a third oligonucleotide hairpin, and a fourth reservoir 516D containing a fourth oligonucleotide hairpin. As described above, each of the oligonucleotide hairpins includes a loop region that contains a payload region encoding arbitrary information, a stem region, and an overhang region. Although four different reservoirs containing oligonucleotide hairpins 516A-D are illustrated in FIG. 5, there may be a greater number of reservoirs 516 if there are more than four different species of oligonucleotide hairpins.

The synthesizer 506 may also include a second set of reservoirs containing invading strands 518. The invading strands may be any of the invading strands described in this disclosure such as the invading strands 120B-E shown in FIG. 3. Each unique species of invading strand may be stored in a separate reservoir 518A-D. Thus, the synthesizer 506 includes a first reservoir 518A containing a first invading strand, a second reservoir 518B containing a second invading strand, a third reservoir 518C containing a third invading strand, and a fourth reservoir 518D containing a fourth invading strand. As described above, each of the invading strands hybridizes to portions of a corresponding oligonucleotide hairpin other than to the overhang region and the overhang side of the stem region. Because each oligonucleotide hairpin has a corresponding invading strand used to open the hairpin, there will typically be the same number of reservoirs containing invading strands 518 as reservoirs containing oligonucleotide hairpins 516. If a different invading strand is used for opening the hairpins hybridized to the initial anchor strand (e.g., invading strand 120A shown in FIG. 1A), then there may be a greater number of reservoirs 518 for storing invading strands than there are reservoirs containing oligonucleotide hairpins 516.

The oligonucleotide hairpins and the invading strands in the respective reservoirs 516 and 518 may be pre-made using any oligonucleotide synthesis technique such as phosphoramidite synthesis or cloning in bacteria. The oligonucleotide hairpins and invading strands may be stored in their respective reservoirs 516 and 518 where they are available to be transferred by fluid delivery pathways 514 to the reaction chamber 510. The oligonucleotides in the reservoirs 516 and 518 may be stored in an aqueous solution that uses a standard buffer for storing oligonucleotides. The concentration of oligonucleotide complexes in the reservoirs 516 and 518 may be, for example, about 0.5, 1, 2, 3, 4, 5, 10, 15, 20, 30, or 50 nM.

Each of the reservoirs 516A, 516B, 516C, 516D containing a species of oligonucleotide hairpin may have a separate fluid delivery pathway 514A, 514B, 514C, 514D to move the respective oligonucleotide hairpins into the reaction chamber 510. Similarly, each of the reservoirs 518A, 518B, 518C, 518D containing a species of invading strand may have a separate fluid delivery pathway 514E, 514F, 514G, 514H to move the respective invading strands into the reaction chamber 510.

One or more of a wash buffer 520, ligase 522, anchor strands 524, and other reagent(s) 526 may also be available in reservoirs connected to the reaction chamber 510 by respective fluid delivery pathways 514I, 514J, 514K, 514L. The wash buffer 520 may include any wash buffer suitable for washing or manipulating oligonucleotides such as TE, TAE, and TBE. The wash buffer may be an aqueous buffer solution or mixed aqueous/organic solvent. Examples of organic solvents that may be added to a wash buffer include polar, miscible organic cosolvents (e.g., DMSO, acetonitrile, etc.) which may help remove metal ions, organic residues, and denatured protein. The reservoir containing ligase 522 may include DNA ligase and/or RNA ligase in appropriate buffer concentration for use in closing nicks in oligonucleotides within the reaction chamber 510.

A reservoir containing anchor strands 524 may provide anchor strands for coating of the substrate 104 in the reaction chamber 510. However, the reservoir of anchor strands 524 may be omitted if substrates 104 are prepared separately and added to the synthesizer 506 with anchor strands attached 106. There may also be one or more additional pools or reservoirs that contain one or more other reagent(s) 526 such as intercalating fluorescent dyes used to detect double-stranded oligonucleotides.

The control circuitry 512 may be configured to selectively open the various fluid delivery pathways 514A-L in response to instructions 508 indicating an order of arbitrary information to encode in one or more oligonucleotides. Thus, the control circuitry 512 can control the order and sequence that contents of the reservoirs containing oligonucleotide hairpins 516 and the reservoirs containing invading strands 518 are added to the reaction chamber 510. This in turn controls the order that various payload regions, with the corresponding arbitrary information, are added to an oligonucleotide. Additionally, the control circuitry 512 may control the addition of other reagents such as ligase and wash buffer. For example, the control circuitry 512 may open the fluid delivery pathways 514A-L in an order that implements a process such as the process 400 illustrated in FIG. 4 or a similar process.

### Illustrative Computer Architecture

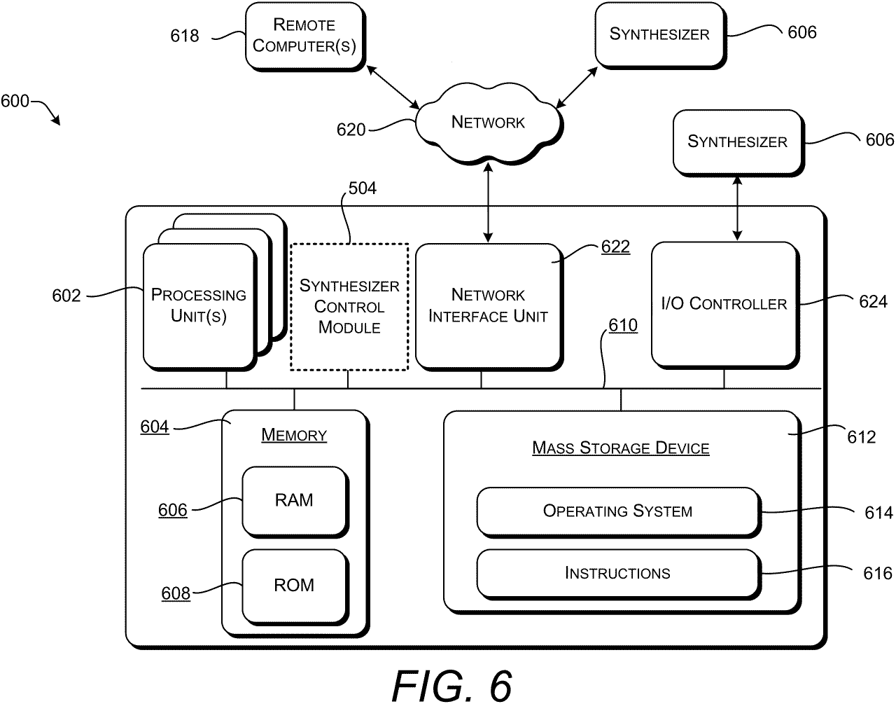

FIG. 6 is a computer architecture diagram showing an illustrative computer hardware and software architecture for a computing device such as the computing device 502 introduced FIG. 5. In particular, the computer 600 illustrated in FIG. 6 can be utilized to implement the synthesizer control module 504.

The computer 600 includes one or more processing units 602, a system memory 604, including a random-access memory 606 (“RAM”) and a read-only memory (“ROM”) 608, and a system bus 610 that couples the memory 604 to the processing unit(s) 602. A basic input/output system (“BIOS” or “firmware”) containing the basic routines that help to transfer information between elements within the computer 600, such as during startup, can be stored in the ROM 608. The computer 600 further includes a mass storage device 612 for storing an operating system 614 and other instructions 616 that represent application programs and/or other types of programs such as, for example, instructions to implement the synthesizer control module 504. The mass storage device 612 can also be configured to store files, documents, and data.

The mass storage device 612 is connected to the processing unit(s) 602 through a mass storage controller (not shown) connected to the system bus 610. The mass storage device 612 and its associated computer-readable media provide non-volatile storage for the computer 600. Although the description of computer-readable media contained herein refers to a mass storage device, such as a hard disk, CD-ROM drive, DVD-ROM drive, or USB storage key, it should be appreciated by those skilled in the art that computer-readable media can be any available computer-readable storage media or communication media that can be accessed by the computer 600.

Communication media includes computer-readable instructions, data structures, program modules, or other data in a modulated data signal such as a carrier wave or other transport mechanism and includes any delivery media. The term “modulated data signal” means a signal that has one or more of its characteristics changed or set in a manner so as to encode information in the signal. By way of example, and not limitation, communication media includes wired media such as a wired network or direct-wired connection, and wireless media such as acoustic, radio frequency, infrared, and other wireless media. Combinations of any of the above should also be included within the scope of computer-readable media.

By way of example, and not limitation, computer-readable storage media can include volatile and non-volatile, removable and non-removable media implemented in any method or technology for storage of information such as computer-readable instructions, data structures, program modules, or other data. For example, computer-readable storage media includes, but is not limited to, RAM 606, ROM 608, EPROM, EEPROM, flash memory or other solid-state memory technology, CD-ROM, digital versatile disks (“DVD”), HD-DVD, BLU-RAY, 4K Ultra BLU-RAY, or other optical storage, magnetic cassettes, magnetic tape, magnetic disk storage or other magnetic storage devices, or any other medium that can be used to store the desired information and which can be accessed by the computer 600. For purposes of the claims, the phrase “computer-readable storage medium,” and variations thereof, does not include waves or signals per se or communication media.

According to various configurations, the computer 600 can operate in a networked environment using logical connections to a remote computer(s) 618 through a network 620. The computer 600 can connect to the network 620 through a network interface unit 622 connected to the system bus 610. It should be appreciated that the network interface unit 622 can also be utilized to connect to other types of networks and remote computer systems. The computer 600 can also include an input/output controller 624 for receiving and processing input from a number of other devices, including a keyboard, mouse, touch input, an electronic stylus (not shown), or equipment such as a synthesizer 506 for synthesizing oligonucleotides. Similarly, the input/output controller 624 can provide output to a display screen or other type of output device (not shown).

It should be appreciated that the software components described herein, when loaded into the processing unit(s) 602 and executed, can transform the processing unit(s) 602 and the overall computer 600 from a general-purpose computing device into a special-purpose computing device customized to facilitate the functionality presented herein. The processing unit(s) 602 can be constructed from any number of transistors or other discrete circuit elements, which can individually or collectively assume any number of states. More specifically, the processing unit(s) 602 can operate as a finite-state machine, in response to executable instructions contained within the software modules disclosed herein. These computer-executable instructions can transform the processing unit(s) 602 by specifying how the processing unit(s) 602 transitions between states, thereby transforming the transistors or other discrete hardware elements constituting the processing unit(s) 602.

Encoding the software modules presented herein can also transform the physical structure of the computer-readable media presented herein. The specific transformation of physical structure depends on various factors, in different implementations of this description. Examples of such factors include, but are not limited to, the technology used to implement the computer-readable media, whether the computer-readable media is characterized as primary or secondary storage, and the like. For example, if the computer-readable media is implemented as semiconductor-based memory, the software disclosed herein can be encoded on the computer-readable media by transforming the physical state of the semiconductor memory. For instance, the software can transform the state of transistors, capacitors, or other discrete circuit elements constituting the semiconductor memory. The software can also transform the physical state of such components to store data thereupon.

As another example, the computer-readable media disclosed herein can be implemented using magnetic or optical technology. In such implementations, the software presented herein can transform the physical state of magnetic or optical media, when the software is encoded therein. These transformations can include altering the magnetic characteristics of particular locations within given magnetic media. These transformations can also include altering the physical features or characteristics of particular locations within given optical media, to change the optical characteristics of those locations. Other transformations of physical media are possible without departing from the scope and spirit of the present description, with the foregoing examples provided only to facilitate this discussion.

In light of the above, it should be appreciated that many types of physical transformations take place in the computer 600 to store and execute the software components presented herein. It also should be appreciated that the architecture shown in FIG. 6 for the computer 600, or a similar architecture, can be utilized to implement many types of computing devices such as desktop computers, notebook computers, servers, supercomputers, gaming devices, tablet computers, and other types of computing devices known to those skilled in the art. For example, the computer 600 may be wholly or partially integrated into the synthesizer 506. It is also contemplated that the computer 600 might not include all of the components shown in FIG. 6, can include other components that are not explicitly shown in FIG. 6, or can utilize an architecture completely different than that shown in FIG. 6.

### Illustrative Embodiments

The following clauses described multiple possible embodiments for implementing the features described in this disclosure. The various embodiments described herein are not limiting nor is every feature from any given embodiment required to be present in another embodiment. Any two or more of the embodiments may be combined together unless context clearly indicates otherwise. As used herein in this document “or” means and/or. For example, “A or B” means A without B, B without A, or A and B. As used herein, “comprising” means including all listed features and potentially including addition of other features that are not listed. “Consisting essentially of” means including the listed features and those additional features that do not materially affect the basic and novel characteristics of the listed features. “Consisting of” means only the listed features to the exclusion of any feature not listed.

Embodiment 1. A method of assembling an oligonucleotide encoding arbitrary information (124), the method comprising: a. contacting an anchor strand (106) attached to a substrate (104) with an oligonucleotide hairpin (102A) under conditions such that an overhang region (112) of the oligonucleotide hairpin hybridizes to a distal portion (114) of the anchor strand leaving a proximal portion (116) of the anchor strand as a toehold region (122), the oligonucleotide hairpin comprising a loop region (110) that includes a payload region (118A) which encodes arbitrary information; b. contacting the oligonucleotide (124) with a ligase under conditions such that the ligase catalyzes formation of a phosphodiester bond between the oligonucleotide hairpin and the anchor strand; and c. contacting the oligonucleotide with an invading strand (120) under conditions such that the invading strand hybridizes to the toehold region of the anchor strand, the distal portion of the anchor strand, and a portion of the oligonucleotide hairpin including the payload region thereby opening the hairpin structure.

Embodiment 2. The method of embodiment 1, further comprising repeating steps a-c with an additional oligonucleotide hairpin (102B) that includes an additional payload region (118B) which encodes additional arbitrary information.

Embodiment 3. The method of embodiment 2, further comprising releasing a doublestranded structure formed from the oligonucleotide hairpins and the invading strands from the substrate.

Embodiment 4. The method of any of embodiments 1-3, wherein a length of the payload region is 1-5 nucleotides, a length of the overhang region is at least 3 nucleotides, and a length of the toehold region is at least 6 nucleotides.

Embodiment 5. The method of any of embodiments 1-4, wherein the substrate is a microelectrode array (202) and the method further comprises selectively activating at least one electrode (204) in the microelectrode array to electrostatically attract the oligonucleotide hairpin to a specific location on the surface of the substrate.

Embodiment 6. The method of embodiment 5, further comprising assembling a first oligonucleotide having a first arbitrary information at a first location on the substrate and assembling a second oligonucleotide encoding second arbitrary information a second location on the substrate.

Embodiment 7. The method of any of embodiments 1-6, further comprising: identifying a string of binary digits to encode in the oligonucleotide; repeating steps a-c adding during each round an oligonucleotide hairpin that encodes a first bit or a second bit, the oligonucleotide hairpins added in an order based on the string of binary digits, wherein the overhang region of each oligonucleotide hairpin hybridizes to nucleotides that were the overhang region of a previously added oligonucleotide hairpin; and determining that the entire string of the binary digits is encoded in the oligonucleotide.

Embodiment 8. The method of embodiment 7, further comprising releasing the oligonucleotide from the substrate.

Embodiment 9. A plurality of oligonucleotides encoding at least a first arbitrary information and a second arbitrary information, the plurality of oligonucleotides comprising: a first oligonucleotide hairpin (102A) comprising a first loop region (110) that contains a first payload region (118A) encoding the first arbitrary information, a first stem region (108), and a first overhang region (112); a first invading strand (120C) that hybridizes to portions of the first oligonucleotide hairpin other than to the first overhang region (112) and an overhang side (302)of the first stem region; a second oligonucleotide hairpin (102C) comprising a second loop region (110) that contains the first payload region (118A) encoding the first arbitrary information, a second stem region (108), and a second overhang region (112) that hybridizes to the first overhang region (112); a second invading strand (120D) that hybridizes to portions of the second oligonucleotide hairpin (102C) other than to the second overhang region (112) and an overhang side (302) of the second stem region; a third oligonucleotide hairpin (102D) comprising a third loop region (110) that contains a second payload region (118B) encoding the second arbitrary information, the first stem region (108), and the first overhang region (112) that hybridizes to the second overhang region (112); a third invading strand (120E) that hybridizes to portions of the third oligonucleotide hairpin other than to the first overhang region (112) and the overhang side (302) of the first stem region; a fourth oligonucleotide hairpin (102B) comprising a fourth loop region (110) that contains the second payload region (118B) encoding the second arbitrary information, the second stem region (108), and the second overhang region (112) that hybridizes to the first overhang region (112); and a fourth invading strand (120B) that hybridizes to portions of the fourth oligonucleotide hairpin (102B) other than to the second overhang region (112) and the overhang side (302) of the second stem region.

Embodiment 10. The plurality of oligonucleotides of embodiment 9, wherein each of the first oligonucleotide hairpin, the second oligonucleotide hairpin, the third oligonucleotide hairpin, the fourth oligonucleotide hairpin, the first invading strand, the second invading strand, the third invading strand, and the fourth invading strand are present in different containers.

Embodiment 11. The plurality of oligonucleotides of any of embodiments 9-10, wherein a length of the first payload region and the second payload region are each independently 1-5 nucleotides.

Embodiment 12. The plurality of oligonucleotides of any of embodiments 9-11, wherein the first arbitrary information is a first bit value and the second arbitrary information is a second bit value.

Embodiment 13. The plurality of oligonucleotides of any of embodiments 9-12, wherein a length of the first overhang region and the second overhang region are each independently at least 3 nucleotides.

Embodiment 14. The plurality of oligonucleotides of any of embodiments 9-13, wherein a length of a non-overhang side (304) of the first stem region and a non-overhang side of the second stem region are each independently at least 6 nucleotides.

Embodiment 15. The plurality of oligonucleotides of any of embodiments 9-14, wherein the first oligonucleotide hairpin and the second oligonucleotide hairpin have nucleotide sequences such that an end nucleotide on a non-overhang side of the first stem region (306) of the first oligonucleotide is directly adjacent to an end nucleotide on the second overhang region (308) of the second oligonucleotide hairpin when the first overhang region of the first oligonucleotide hairpin is hybridized to the second overhang region of the second oligonucleotide hairpin.

Embodiment 16. The plurality of oligonucleotides of any of embodiments 9-15, wherein the first loop region includes at least one nucleotide that is not part of the first payload region.

Embodiment 17. A system (500) for assembling an oligonucleotide encoding arbitrary information (124), the system comprising: a substrate (104) coated with a plurality of anchor strands (106); a reaction chamber (510) in fluid contact with the substrate; a first fluid delivery pathway (514A) configured to introduce a first oligonucleotide hairpin encoding first arbitrary information into the reaction chamber; a second fluid delivery pathway (514E) configured to introduce a first invading strand that hybridizes to a portion of the first oligonucleotide hairpin into the reaction chamber; a third fluid delivery pathway (514B) configured to introduce a second oligonucleotide hairpin encoding second arbitrary information into the reaction chamber; a fourth fluid delivery pathway (514F) configured to introduce a second invading strand that hybridizes to a portion of the second oligonucleotide hairpin into the reaction chamber; a fifth fluid delivery pathway (514J) configured to introduce ligase into the reaction chamber; a sixth fluid delivery pathway (514I) configured to introduce a wash buffer into the reaction chamber; and control circuitry (512) configured to selectively open the first fluid delivery pathway, the second fluid delivery pathway, the third fluid delivery pathway, the fourth fluid delivery pathway, the fifth fluid delivery pathway, and the sixth fluid delivery pathway in response to instructions (508) indicating an order of the first arbitrary information and the second arbitrary information to encode in the oligonucleotide.

Embodiment 18. The system of embodiment 17, wherein the substrate is a microelectrode array (202) and the control circuitry is further configured to selectively activate individual electrodes (204) in the microelectrode array with a voltage sufficient to attract oligonucleotides to the substrate.

Embodiment 19. The system of any of embodiments 17-18, wherein the control circuitry is configured to cause, through selective activation of individual electrodes in the microelectrode array, the system to assemble a first oligonucleotide encoding a first sequence of arbitrary information at a first location on the substrate and a second oligonucleotide encoding a second sequence of arbitrary information at a second location on the substrate.

Embodiment 20. The system of any of embodiments 17-19, further comprising: a first reservoir (516) containing the first oligonucleotide hairpin, wherein the first oligonucleotide hairpin comprises a first loop region that contains a first payload region encoding the first arbitrary information, a first stem region, and a first overhang region; a second reservoir (518) containing the first invading strand, wherein the first invading strand hybridizes to portions of the first oligonucleotide hairpin other than to the first overhang region and an overhang side of the first stem region; a third reservoir (516) containing the second oligonucleotide hairpin, wherein the second oligonucleotide hairpin comprises a second loop region that contains a second payload region encoding the second arbitrary information, a second stem region, and a second overhang region; and a fourth reservoir (518) containing the second invading strand, wherein the second invading strand hybridizes to portions of the second oligonucleotide hairpin other than to the second overhang region and an overhang side of the second stem region.

### Conclusion

Although the subject matter has been described in language specific to structural features and/or methodological acts, it is to be understood that the subject matter defined in the appended claims is not necessarily limited to the specific features or acts described above. Rather, the specific features and acts are disclosed as example forms of implementing the claims.

The terms “a,” “an,” “the” and similar referents used in the context of describing the invention are to be construed to cover both the singular and the plural unless otherwise indicated herein or clearly contradicted by context. The terms “based on,” “based upon,” and similar referents are to be construed as meaning “based at least in part” which includes being “based in part” and “based in whole,” unless otherwise indicated or clearly contradicted by context. The terms “portion,” “part,” or similar referents are to be construed as meaning at least a portion or part of the whole including up to the entire noun referenced. As used herein, “approximately” or “about” or similar referents denote a range of ± 10% of the stated value.

Certain embodiments are described herein, including the best mode known to the inventors for carrying out the invention. Of course, variations on these described embodiments will become apparent to those of ordinary skill in the art upon reading the foregoing description. Skilled artisans will know how to employ such variations as appropriate, and the embodiments disclosed herein may be practiced otherwise than specifically described. Accordingly, all modifications and equivalents of the subject matter recited in the claims appended hereto are included within the scope of this disclosure. Moreover, any combination of the above-described elements in all possible variations thereof is encompassed by the invention unless otherwise indicated herein or otherwise clearly contradicted by context.

Furthermore, references have been made to publications, patents and/or patent applications throughout this specification. Each of the cited references is individually incorporated herein by reference for its particular cited teachings as well as for all that it discloses.

1. A method of assembling an oligonucleotide encoding arbitrary information, the method comprising:
a. contacting an anchor strand attached to a substrate with an oligonucleotide hairpin under conditions such that an overhang region of the oligonucleotide hairpin hybridizes to a distal portion of the anchor strand leaving a proximal portion of the anchor strand as a toehold region, the oligonucleotide hairpin comprising a loop region that includes a payload region which encodes arbitrary information;
b. contacting the oligonucleotide with a ligase under conditions such that the ligase catalyzes formation of a phosphodiester bond between the oligonucleotide hairpin and the anchor strand; and
c. contacting the oligonucleotide with an invading strand under conditions such that the invading strand hybridizes to the toehold region of the anchor strand, the distal portion of the anchor strand, and a portion of the oligonucleotide hairpin including the payload region thereby opening the hairpin structure.

2. The method of claim 1, further comprising repeating steps a-c with an additional oligonucleotide hairpin that includes an additional payload region which encodes additional arbitrary information.

3. The method of claim 2, further comprising releasing a double-stranded structure formed from the oligonucleotide hairpins and the invading strands from the substrate.

4. The method of claim 1, wherein a length of the payload region is 1-5 nucleotides, a length of the overhang region is at least 3 nucleotides, and a length of the toehold region is at least 6 nucleotides.

5. The method of claim 1, wherein the substrate is a microelectrode array and the method further comprises selectively activating at least one electrode in the microelectrode array to electrostatically attract the oligonucleotide hairpin to a specific location on the surface of the substrate.

6. The method of claim 5, further comprising assembling a first oligonucleotide having a first arbitrary information at a first location on the substrate and assembling a second oligonucleotide encoding second arbitrary information a second location on the substrate.

7. The method of claim 1, further comprising:
identifying a string of binary digits to encode in the oligonucleotide;
repeating steps a-c adding during each round an oligonucleotide hairpin that encodes a first bit or a second bit, the oligonucleotide hairpins added in an order based on the string of binary digits, wherein the overhang region of each oligonucleotide hairpin hybridizes to nucleotides that were the overhang region of a previously added oligonucleotide hairpin; and
determining that the entire string of the binary digits is encoded in the oligonucleotide.

8. The method of claim 7, further comprising releasing the oligonucleotide from the substrate.

9. A plurality of oligonucleotides encoding at least a first arbitrary information and a second arbitrary information, the plurality of oligonucleotides comprising:
a first oligonucleotide hairpin comprising a first loop region that contains a first payload region encoding the first arbitrary information, a first stem region, and a first overhang region;
a first invading strand that hybridizes to portions of the first oligonucleotide hairpin other than to the first overhang region and an overhang side of the first stem region;
a second oligonucleotide hairpin comprising a second loop region that contains the first payload region encoding the first arbitrary information, a second stem region, and a second overhang region that hybridizes to the first overhang region;
a second invading strand that hybridizes to portions of the second oligonucleotide hairpin other than to the second overhang region and an overhang side of the second stem region;
a third oligonucleotide hairpin comprising a third loop region that contains a second payload region encoding the second arbitrary information, the first stem region, and the first overhang region that hybridizes to the second overhang region;
a third invading strand that hybridizes to portions of the third oligonucleotide hairpin other than to the first overhang region and the overhang side of the first stem region;
a fourth oligonucleotide hairpin comprising a fourth loop region that contains the second payload region encoding the second arbitrary information, the second stem region, and the second overhang region that hybridizes to the first overhang region; and
a fourth invading strand that hybridizes to portions of the fourth oligonucleotide hairpin other than to the second overhang region and the overhang side of the second stem region.

10. The plurality of oligonucleotides of claim 9, wherein each of the first oligonucleotide hairpin, the second oligonucleotide hairpin, the third oligonucleotide hairpin, the fourth oligonucleotide hairpin, the first invading strand, the second invading strand, the third invading strand, and the fourth invading strand are present in different containers.

11. The plurality of oligonucleotides of claim 9, wherein a length of the first payload region and the second payload region are each independently 1-5 nucleotides.

12. The plurality of oligonucleotides of claim 9, wherein the first arbitrary information is a first bit value and the second arbitrary information is a second bit value.

13. The plurality of oligonucleotides of claim 9, wherein a length of the first overhang region and the second overhang region are each independently at least 3 nucleotides.

14. The plurality of oligonucleotides of claim 9, wherein a length of a non-overhang side of the first stem region and a non-overhang side of the second stem region are each independently at least 6 nucleotides.

15. The plurality of oligonucleotides of claim 9, wherein the first oligonucleotide hairpin and the second oligonucleotide hairpin have nucleotide sequences such that an end nucleotide on a non-overhang side of the first stem region of the first oligonucleotide is directly adjacent to an end nucleotide on the second overhang region of the second oligonucleotide hairpin when the first overhang region of the first oligonucleotide hairpin is hybridized to the second overhang region of the second oligonucleotide hairpin.

16. The plurality of oligonucleotides of claim 9, wherein the first loop region includes at least one nucleotide that is not part of the first payload region.

17. A system for assembling an oligonucleotide encoding arbitrary information, the system comprising:
a substrate coated with a plurality of anchor strands;
a reaction chamber in fluid contact with the substrate;
a first fluid delivery pathway configured to introduce a first oligonucleotide hairpin encoding first arbitrary information into the reaction chamber;
a second fluid delivery pathway configured to introduce a first invading strand that hybridizes to a portion of the first oligonucleotide hairpin into the reaction chamber;
a third fluid delivery pathway configured to introduce a second oligonucleotide hairpin encoding second arbitrary information into the reaction chamber;
a fourth fluid delivery pathway configured to introduce a second invading strand that hybridizes to a portion of the second oligonucleotide hairpin into the reaction chamber;
a fifth fluid delivery pathway configured to introduce ligase into the reaction chamber;
a sixth fluid delivery pathway configured to introduce a wash buffer into the reaction chamber; and
control circuitry configured to selectively open the first fluid delivery pathway, the second fluid delivery pathway, the third fluid delivery pathway, the fourth fluid delivery pathway, the fifth fluid delivery pathway, and the sixth fluid delivery pathway in response to instructions indicating an order of the first arbitrary information and the second arbitrary information to encode in the oligonucleotide.

18. The system of claim 17, wherein the substrate is a microelectrode array and the control circuitry is further configured to selectively activate individual electrodes in the microelectrode array with a voltage sufficient to attract oligonucleotides to the substrate.

19. The system of claim 18, wherein the control circuitry is configured to cause, through selective activation of individual electrodes in the microelectrode array, the system to assemble a first oligonucleotide encoding a first sequence of arbitrary information at a first location on the substrate and a second oligonucleotide encoding a second sequence of arbitrary information at a second location on the substrate.

20. The system of claim 17, further comprising:
a first reservoir containing the first oligonucleotide hairpin, wherein the first oligonucleotide hairpin comprises a first loop region that contains a first payload region encoding the first arbitrary information, a first stem region, and a first overhang region;
a second reservoir containing the first invading strand, wherein the first invading strand hybridizes to portions of the first oligonucleotide hairpin other than to the first overhang region and an overhang side of the first stem region;
a third reservoir containing the second oligonucleotide hairpin, wherein the second oligonucleotide hairpin comprises a second loop region that contains a second payload region encoding the second arbitrary information, a second stem region, and a second overhang region; and
a fourth reservoir containing the second invading strand, wherein the second invading strand hybridizes to portions of the second oligonucleotide hairpin other than to the second overhang region and an overhang side of the second stem region.
